SEQUENCE LISTING (8 sequences)

SEQ ID NO: 1 - DNA, 20 bp, Artificial Sequence
  Synthetic Oligonucleotide
aagaggctaagaccgccttc

SEQ ID NO: 2 - DNA, 20 bp, Artificial Sequence
  Synthetic Oligonucleotide
cctctggcgaagaaactctg

SEQ ID NO: 3 - DNA, 20 bp, Artificial Sequence
  Synthetic Oligonucleotide
cactcctgccctctgacttc

SEQ ID NO: 4 - DNA, 20 bp, Artificial Sequence
  Synthetic Oligonucleotide
agagggtgtggagctgctta

SEQ ID NO: 5 - DNA, 20 bp, Artificial Sequence
  Synthetic Oligonucleotide
tgacgataccggagccaatg

SEQ ID NO: 6 - DNA, 20 bp, Artificial Sequence
  Synthetic Oligonucleotide
tcaaaggacttgcccagttt

SEQ ID NO: 7 - DNA, 19 bp, Artificial Sequence
  Synthetic Oligonucleotide
gtggatgccgcctttaact

SEQ ID NO: 8 - DNA, 20 bp, Artificial Sequence
  Synthetic Oligonucleotide
ggaaagccaggatccatttt

(19) United States
(12) Patent Application Publication
(10) Pub. No.: US 2008/0045463 A1
(43) Pub. Date: Feb. 21, 2008

(54) Methods For Lowering Hif-1 Mediated Gene Expression

(76) Inventors: Ajay Verma, Harleysville, PA (US); Huasheng Lu, Boyds, MD (US)

Correspondence Address: MORGAN LEWIS & BOCKIUS LLP, 1111 PENNSYLVANIA AVENUE NW, WASHINGTON, DC, 20004, US

(21) Appl. No.: 11/666,229

(22) PCT Filed: Oct. 25, 2005
(86) PCT No.: PCT/US05/38317

§ 371 Date: Sep. 27, 2007

Related U.S. Application Data
(60) Provisional application No. 60/621,091, filed on Oct. 25, 2004.

(51) Int. Cl. A61K 31/195; A61K 31/34; A61K 31/415; A61K 38/00; A61P 43/00

(52) U.S. Cl. 514/18; 514/399; 514/474; 514/562

(57) ABSTRACT

The present invention relates to the elucidation of specific molecular features of endogenous amino acids and their derivatives for inhibiting hypoxia-inducible gene expression by preventing inactivation of hypoxia-inducible factor hydroxylating enzymes. This invention encompasses agents that can be used to reduce tissue vascularization, cancer cell survival, and to treat obesity.

### GOVERNMENT SUPPORT

The present invention arose in part from research funded by federal grants NS37814 from the National Institution of Health and MDA-905-02-2-0005 and MDA-905-03-2-0001 from the Department of Defense.

### FIELD OF THE INVENTION

The invention relates generally to the changes in gene expression in human tissues, which bring about improved survival of cells in conditions of reduced oxygen supply. The invention relates specifically to the pharmacological inhibition of hypoxia-inducible gene expression by ascorbate, cystine, cysteine, histidine, glutathione and their derivatives.

### BACKGROUND OF THE INVENTION

Hypoxia-stimulated gene products promote glucose uptake, enhance anaerobic glucose metabolism, and induce several cell survival mechanism. The ability to regulate gene expression by hypoxia depends upon a transcription factor known as “hypoxia inducible factor-1” or HIF-1. This transcription factor can activate over four dozen genes that promote cell survival (Table 1). Pharmacological approaches that allow control over cellular HIF-1 activity are hotly pursued because they can impact the three leading cause of death in our society: heart attacks, cancer and stroke. Upregulation of HIF-1 can reduce damage from stroke and heart attacks. Cancer, however, is aggravated by elevated HIF-1 levels. This is because HIF-1 activation also promotes the survival of cancer cells and induces angiogenesis.

The mechanisms by which hypoxia activates HIF-1 to turn on gene expression is rapidly becoming clarified. Two different proteins called HIF-1 alpha (HIF-1α) and HIF-1 beta (HIF-1β) make up this transcription factor and the level of the HIF-1α component is specifically regulated by oxygen tensions (FIG. 1). The regulation of HIF-1α levels involves a novel oxygen sensing mechanism, which directly controls the degradation of the HIF-1α protein (FIG. 1). Both HIF-1α and HIF-11 are constitutively synthesized in most cells of the body. However, the HIF-1α protein is continuously degraded in the presence of oxygen. A newly discovered family of enzymes known as HIF-1α-prolyl hydroxylases (HPHs) regulates the oxygen dependent degradation of HIF-1α. These enzymes catalyze the oxygen-dependent hydroxylation of a key proline residue in the HIF-1α protein. This modification, in turn, directs the ubiquitination and proteasomal degradation of the HIF-1 protein. Another recently identified HIF-1α asparagine hydroxylase enzymatic activity also appears to be involved in inhibiting the transcriptional activation ability of HIF-1 under normal oxygen tensions. The HIF-1α asparagine hydroxylases has been termed Factor Inhibiting HIF or FIH-1.

All of the HPHs and FIH-1 require several co-factors for their activity: oxygen, iron, ascorbate, and 2-oxoglutarate. In the absence of oxygen therefore, HIF-1α is not hydroxylated or degraded, and as a result, its concentration increases dramatically. This allows the HIF-1α and HIF-1β subunits to dimerize, translocate to the nucleus and activate the transcription of several genes that promote survival under low oxygen levels (FIG. 1). The discovery of the HPH enzyme mechanism also explains why iron chelators such as desferrioxamine (DFO) can activate HIF-1 and turn on genes similar to those induced by hypoxia. Molecular interactions at the other cofactor sites have also been shown to regulate HPH activity, HIF-1α levels, and the expression of hypoxia-inducible genes. Thus, artificial analogs of 2-oxoglutarate, such as N-oxalylglycine (NOG) or the cell permeant dimethyloxalylglycine (DMOG) have been shown to block the activity of the HPHs and FIH-1 and thus allow activation of HIF mediated gene expression. However, these artificial 2-oxoglutarate analogs are not specific in inhibiting HPHs or FIH-1 as they were initially designed to inhibit procollagen proline hydroxylases, the enzymes involved in collagen synthesis.

The HPHs, FIH-1 and procollagen proline hydroxylases all belong to the large class of enzymes know as iron and 2-oxoglutarate dependent dioxygenases. These enzymes occur widely in nature and perform valuable biological hydroxylations. One peculiarity of these enzymes is that they are syn-catalytically inactivated. This means that as a result of catalyzing iron mediated oxidations, these enzymes either become oxidized at critical amino acid residues over time or the redox state of the iron becomes useless in carrying out sustained reaction cycles. This syn-catalytic inactivation can be prevented and or reversed by ascorbate. Many cell lines have recently been shown to express significant HIF-1α protein levels and HIF-mediated gene expression in the absence of hypoxia and this is reversible by ascorbate. This suggests that, in many cells, HPHs and FIH-1 may exist in an inactivated form or may be made inactive by some mechanism that is ascorbate reversible. So far, no clear understanding of this phenomenon has been achieved and no pharmaceutical approach has been developed to take advantage of potential mechanisms that activate HPH and FIH-1. Glucose metabolism generates 2-oxoacids, such as pyruvate and oxaloacetate, that are structurally related to 2-oxoglutarate (FIG. 1D) and it was recently demonstrated that the high basal expression of HIF-1α in human gliomas and other cancer cells is in fact dependent upon generation of such 2-oxoacids from glucose metabolism.

A positive correlation between fresh culture medium replacement and HIF-1α protein levels has been observed. By switching from culture medium to the relatively simple Krebs buffer the dependency of HIF-1 at levels on the metabolism of glucose to pyruvate was determined. In addition, it was also determined that the glucose metabolites pyruvate and oxaloacetate are responsible for maintaining elevated HIF-1 levels in cancer cells.

Other molecules in the art that have been shown to inhibit HIF activity (see, for example, U.S. Patent Applications 20050215503 and 20050096370). These molecules act either directly on the HIF transcription factor or effect HIF-1α expression. These molecules have a global effect on the HIF transcription factor, shutting down all HIF expressed genes. Such global regulation can be toxic to normal mammalian cells. The agents and methods disclosed herein regulate HIF activity with regard to hydroxylation and degradation of HIF-1 α (see FIG. 7). These agents selectively lower HIF-1 activation by the 2-oxoacid glucose metabolites but not by HIF activation by hypoxia. Thus, the agents and methods of the present invention will regulate HIF in a more directed fashion and should be less toxic to normal mammalian cells.

### SUMMARY OF THE INVENTION

The present invention relates to the elucidation of specific molecular features of endogenous amino acids and their derivatives for inhibiting hypoxia-inducible gene expression by preventing inactivation of hypoxia-inducible factor hydroxylating enzymes. This invention encompasses agents that can be used to reduce tissue vascularization, cancer cell survival, inflammation, tissue edema, and to treat obesity.

The present invention encompasses methods for inhibiting proliferation of a cancer cell in a mammal diagnosed with cancer, comprising administering to said mammal a composition comprising one or more agents selected from the group consisting of ascorbate, cystine, cysteine, histidine, glutathione and derivatives thereof. In one embodiment, the method comprises an additional cancer therapy. In another embodiment the additional cancer therapy is selected from the group consisting of chemotherapy, radiation therapy, hormonal therapy and immunotherapy.

The present invention also encompasses methods of inhibiting tissue neovascularization in a mammal comprising administering to said mammal a composition comprising one or more agents selected from the group consisting of ascorbate, cystine, cysteine, histidine, glutathione and derivatives thereof. In one embodiment the neovascularization is associated with cancer (e.g. angiogenesis) In another embodiment, the method encompasses combination therapies with additional anti-angiogenic agents such as angiostatin and/or endostatin.

In another embodiment, the neovascularization is associated with inflammatory conditions. In one embodiment, the inflammatory condition is selected from the group consisting of dermatitis, psoriasis and arthritis. In another embodiment, the invention contemplates administering the agents of the invention with at least one additional anti-inflammatory agent.

HIF-1 activity is required for normal cellular immunity. The invention contemplates administering the agents of the invention to lower HIF-1 in immune cells in the setting of chronic inflammation.

In another embodiment, the tissue neovascularization is associated with loss of vision (e.g. macular degeneration (wet and dry), diabetic retinopathy). In one embodiment the loss of vision is caused by retinal neovacularization.

In another embodiment, the tissue edema that accompanies the neovascularization process is targeted. The invention contemplates administering the agents of the invention in order to reduce vasogenic tissue edema following ischemia reperfusion, trauma, or in the setting of metabolic diseases that promote inactivation of HIF-1 hydroxylases.

### BRIEF DESCRIPTION OF FIGURES

FIG. 1: The hypoxia signal transduction pathway

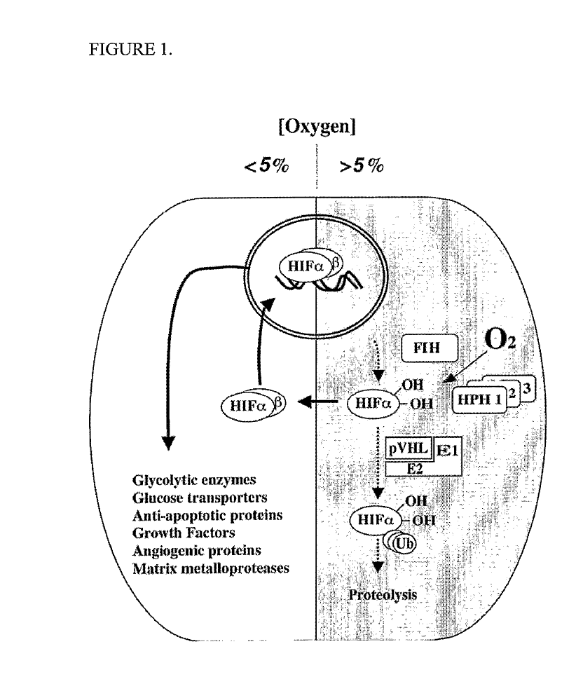

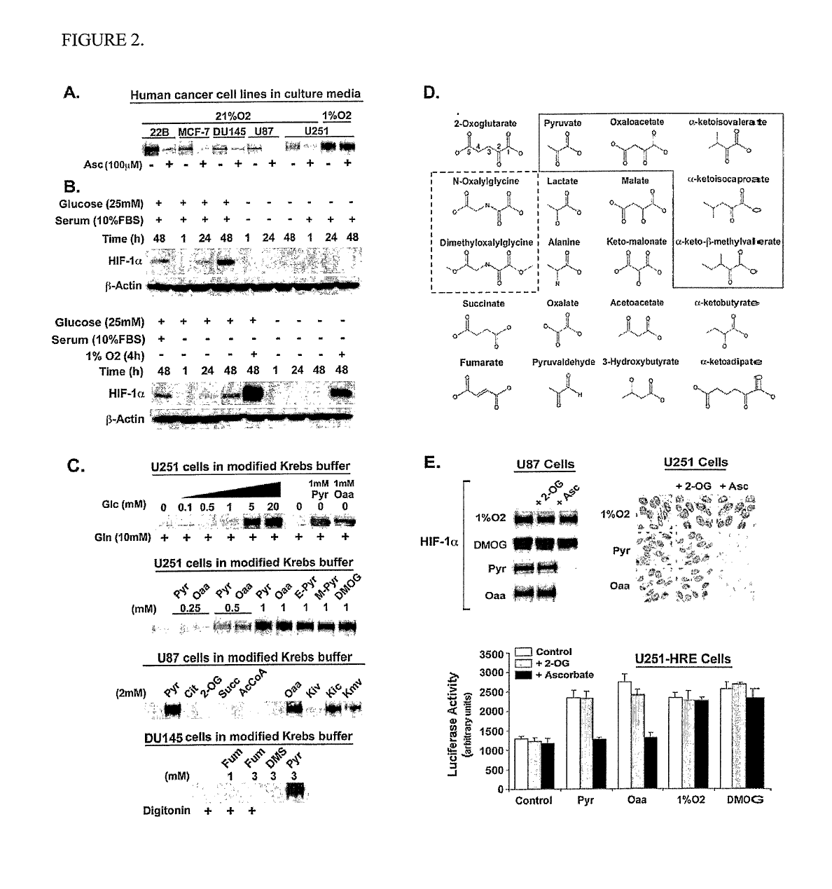

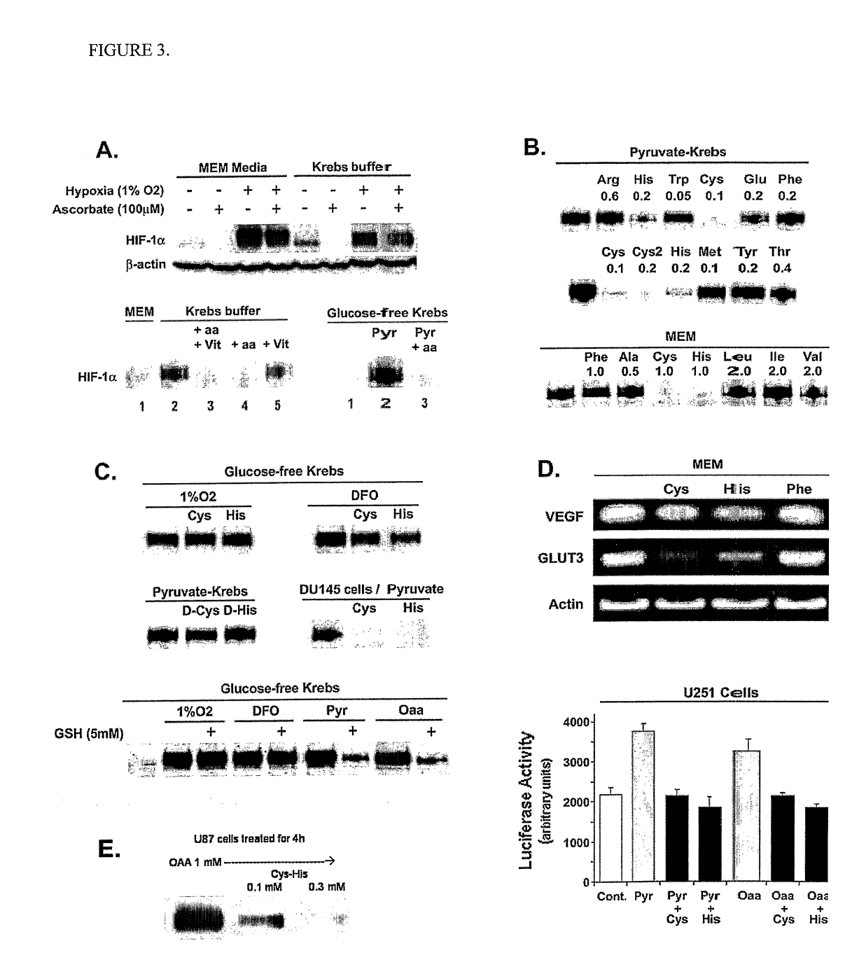

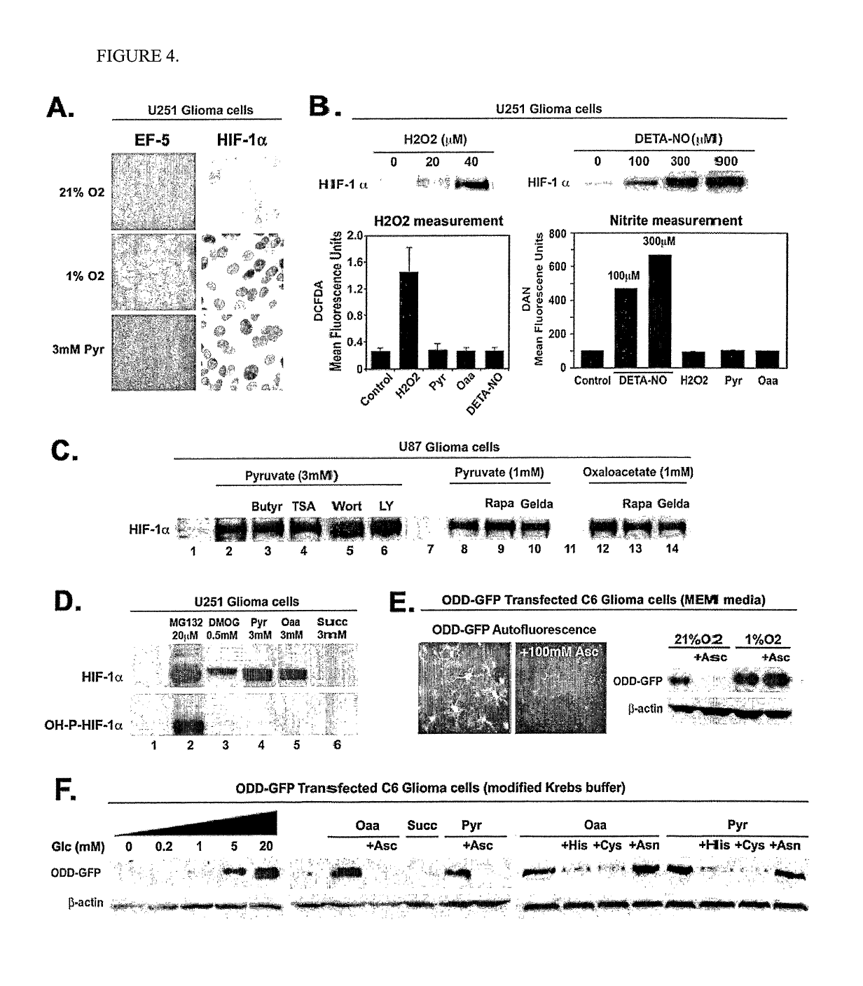

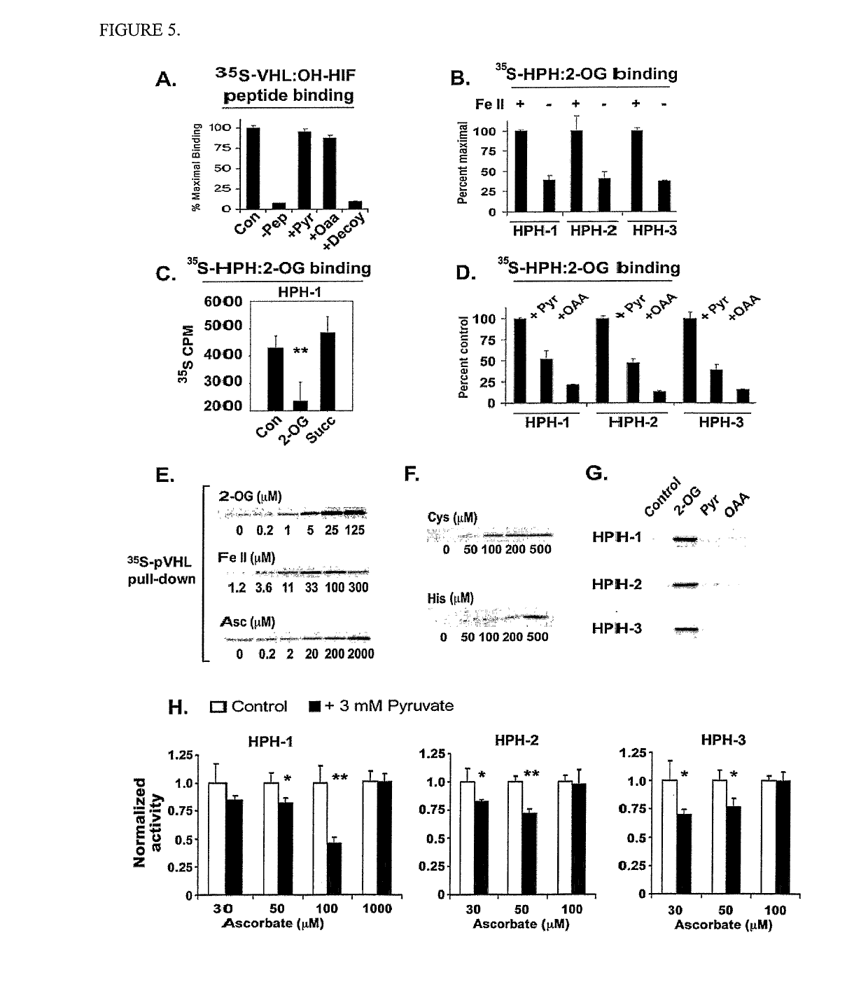

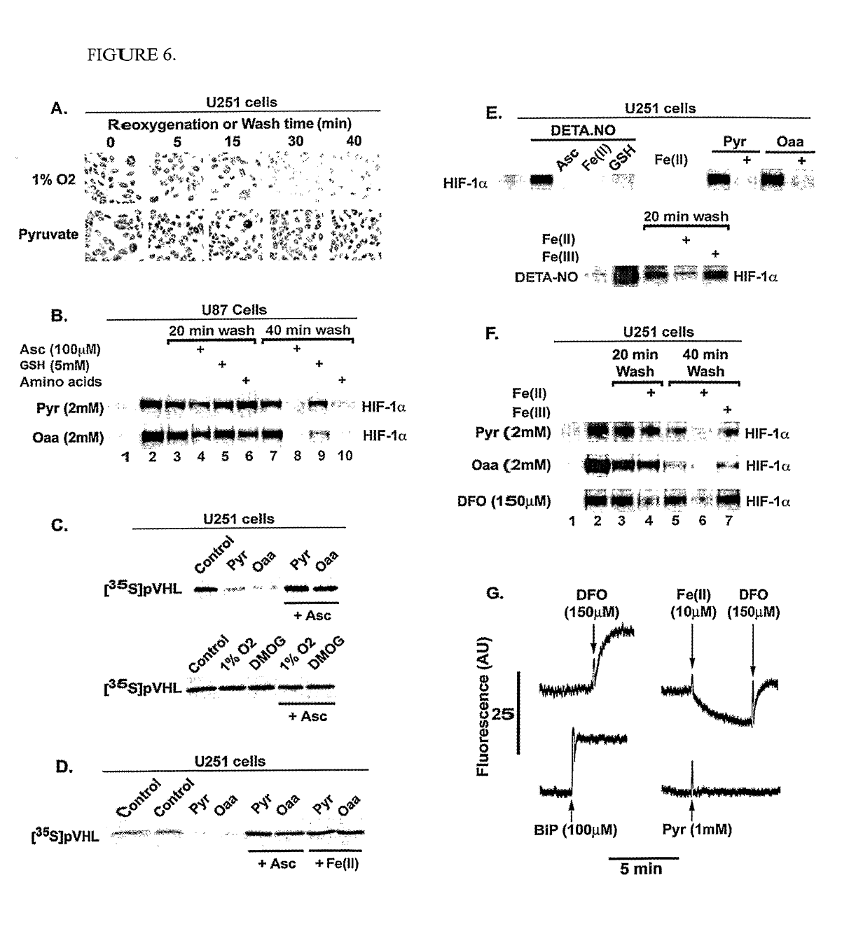

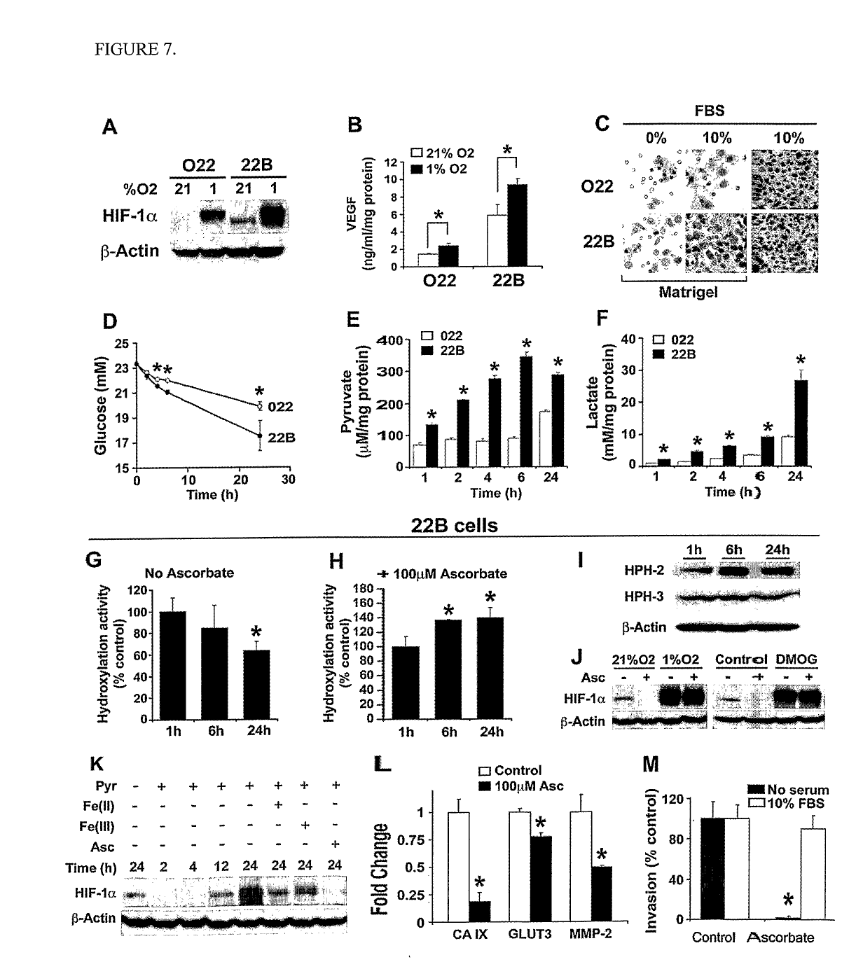

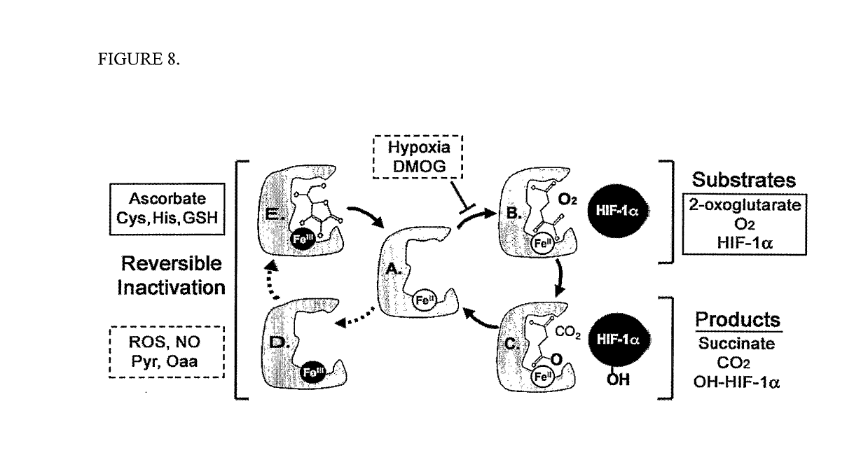

Nearly all cells in multicellular organisms adapt to hypoxia via gene expression controlled by the heterodimeric transcription factor HIF-1. The oxygen regulated HIF-1α subunit is constitutively synthesized in all cells. Under normoxic condition (>5% oxygen, right side of figure), specific HIF-1α hydroxylases incorporate oxygen directly into the HIF-1α protein. Asparaginyl hydroxylation by FIH blocks the transactivating ability of HIF-1α. Proline hydroxylation of HIF-1α by HPH isoforms 1, 2 and 3 generates recognition motifs for binding to the von Hippl Lindau protein (pVHL), which is the E3 component of a ubiqutin ligase complex. Ubiquination and proteolysis of HIF-1α under normoxia assures that HIF-1 responsive genes are not transcribed. Under hypoxia (<8% oxygen, left side of figure) HIF-1α escapes hydroxylation, dimerizes with HIF-1β, translocates to the nucleus, and activates the transcription of several genes that result in enhanced glycolysis, cell survival and angiogenesis. Normally these adaptations lead to improved cell and organism survival. Hypoxic cancer cells also activate gene expression using the same pathway and this may underlie the adverse clinical effects of tumor hypoxia. Moreover, cancer cells display a high basal level of HIF-1 even in the presence of oxygen. This suggests an impairment of HIF-1 degrading activity in cancer cells.

FIG. 2. 2-Oxoacid metabolites induce ascorbate-reversible HIF-1α

(A) Nuclear HIF-1α protein in normoxic human cancer cell lines grown±ascorbate (Asc) for 24 hours in complete media containing 25 mM (22B), 11.1 mM (MCF-7, DU145) or 5.5 mM glucose (U87, U251). (B) Nuclear HIF-1α levels in 22B cells cultured in complete DMEM or glucose-free medium±serum. Hypoxic responsiveness of HIF-1α was tested by exposing cells to 1% O₂ for four hours. (C) U251, U87, and DU145 cells were switched for four hours to Krebs buffer modified as indicated with specific metabolic fuels or intermediary metabolites. Unlabeled lanes represent cells were treated only with glucose-free Krebs buffer. (D) Structures of key metabolic intermediates. Boxes indicate HIF-1α inducing natural metabolites (straight line) and artificial 2-OG analogs (dashed line). (E) The indicated cells were cultured in glucose-free Krebs buffer under hypoxia (1% O₂) or in normoxia with either DMOG (0.5 mM), pyruvate (3 mM), or oxaloacetate (3 mM). Nuclear HIF-1α accumulation was measured at four hours and HRE luciferase activity was determined at 8 h. Cultures also contained 2-OG (10 mM) or ascorbate (100 μM) where indicated. Abbreviations are: AcCoA, acetyl-CoA; Asc, ascorbate; Cit, citrate; DMOG, dimethyloxalylglycine; DMS, dimethylsuccinate; E-Pyr, ethyl-pyruvate; Fum, fumarate; Glc, glucose; Gln, glutamine; Kic, ketoisocaproate; Kiv, ketoisovalerate; Kmv, ketomethylvalerate; M-Pyr, methyl-pyruvate; 2-OG, 2-oxoglutarate: Oaa, oxaloacetate; Pyr, pyruvate; Succ, succinate.

FIG. 3. Cysteine and histidine prevent metabolic HIF-1α accumulation

(A) Nuclear HIF-1α in U87 cells cultured for four hours in either fresh MEM or Krebs buffer. Amino acid (aa) and vitamin (Vit) additives found in MEM were added to Krebs buffer where indicated and pyruvate (1 mM) was substituted for glucose where indicated. (B) Glucose-free Krebs was reconstituted with pyruvate (1 mM) and individual amino acid at concentrations found normally in complete MEM. Nuclear HIF-1α was determined after four hours culture. Alternatively, each amino acid was individually raised to 5× its normal concentration in complete MEM and cells cultured for 2 days. (C) U87 cells were cultured for four hours in glucose-free Krebs buffer containing 150 μM DFO, or 1 mM pyruvate or oxaloacetate. Cells were additionally exposed to L- or D-isomers of cysteine and histidine (250 μM), reduced glutathione (GSH) or 1% O₂ where indicated. One experiment with DU145 cells is also shown. (D) RT-PCR expression of VEGF, GLUT3, and actin in U87 cells following 2d culture in MEM containing 5× cysteine, histidine, or phenylalanine. Luciferase activity was determined in U251-HRE cells cultured in glucose-free Krebs containing either pyruvate or oxaloacetate (2 mM)±cysteine or histidine (250 μM) for 8 h. (E) Demonstration of the effectiveness of cysteine and histidine derivative in lowering normoxic HIF-1α. Cells reated with oxaloacetate induce HIF-1α as shown by this western blot of nuclear extracts. Co-incubation of cells with submillimolar doses of the peptide dimer of cysteine and histidine lowers HIF-1α accumulation.

FIG. 4. Pyruvate and oxaloacetate block oxygen-dependent protein degradation

(A) EF-5 staining in U251 glioma cells. EF-5 (500 μM) was added to the culture medium and cells were then incubated under the indicated conditions for four hours followed by fixation and immunofluorescence staining for EF-5 adducts. (B) Nuclear HIF-1α in U251 cells treated with indicated doses of H₂O₂ or DETA-NO for four hours. Media H₂O₂ and nitrite in cells were determined after four hours culture in glucose-free Krebs (control) supplemented with either 40 μM H₂O₂, 3 mM pyruvate, 3 mM oxaloacetate, or 300 μM DETA-NO. (C) Nuclear HIF-1α levels in U87 grown for four hours in glucose-free media alone (lanes 1,7,11) or with added pyruvate or oxaloacetate in the presence of butyrate (Butyr, 10 mM), trichostatin A (TSA, 300 ng/ml), wortmannin (Wort, 1 μM), LY294002 (LY, 20 μM), rapamycin (Rapa, 100 nM), or geldanamycin (Gelda, 3 μM). (D) HIF-1α hydroxyproline detection. U251 cells were cultured for four hours in MEM (lanes 1,2) or glucose-free Krebs supplemented with the indicated agents. Whole cells extracts were then stained with antibodies recognizing either HIF-1α or the HIF-1α ODD region containing hydroxyproline at P546. (E) GFP expression in ODD-GFP transfected C6 glioma cells was measured by fluorescence microscopy or western blotting. (F) ODD-GFP transfectecd C6 glioma cells were grown for 24 hours in Krebs buffer modified to contain the indicated amounts of glucose, or 2 mM oxaloacetate, succinate, or pyruvate. Ascorbate (100 μM) and the amino acids histidine, cysteine and asparagine (0.5 mM each) were added where indicated.

FIG. 5. Pyruvate and oxaloacetate interact with and inhibit HPHs

(A) Binding of ³⁵S-pVHL to the hydroxylated HIF ODD peptide was measured in the presence or absence of 10 mM pyruvate or oxaloacetate. (B) Binding of the ³⁵S—HPHs to 2-OG sepharose was measured in the presence or absence of 200 μM Fe(II). (C) Binding of ³⁵S-HPH-1 to 2-OG was measured in the presence of free 2-OG or succinate (10 ml each). (D) The binding of ³⁵S-HPH-2 to 2-OG was measured in the presence of pyruvate or oxaloacetate (10 mM). (E) Activity of in vitro translated HPHs was determined in the presence of increasing doses of 2-OG, Fe(II) and ascorbate (Asc) while the other components were kept constant (2 mM 2-OG, 250 μM FeSO₄, 2 mM ascorbate, 1 mM DTT, streptavidin beads pre-incubated with 1 μg biotinylated HIF-1α peptide, 2.5 μl IVT product) or (F) cysteine and histidine, in the absence of ascorbate and DTT. (G) Pyruvate and oxaloacetate (2 mM) could not replace 2-OG as productive substrates. (H) Pyruvate and oxaloacetate reduced enzymatic activity in a specific window of ascorbate concentrations. Quantitative data are means +/−SEM. (* P<0.05, ** P<0.01).

FIG. 6. Reversible inactivation of cellular HIF-1α hydroxylation by 2-oxoacids

(A) U251 cells cultured for four hours in glucose-free Krebs either in 1% O₂ or with 3 mM pyruvate in 21% O₂ were washed (3×) and maintained in oxygenated glucose-free Krebs buffer until formalin fixation and staining for HIF-1α immunoreactivity at the indicated times. (B) U87 were treated in glucose-free Krebs buffer±1 mM pyruvate or oxaloacetate. After four hours, cells were washed in glucose-free Krebs for various times±100 μM ascorbate, 5 mM GSH or the MEM amino acid mixture being included in the wash. (C) U251 were cultured for four hours in glucose-free Krebs buffer±the indicated additions of 2 mM pyruvate, 2 mM oxaloacetate, or 1 mM DMOG or 100 μM ascorbate. After washing, whole cell extracts were prepared and used as the source of HPH enzyme in ³⁵S-pVHL pull down assays with no exogenous addition of iron, or ascorbate. (D) Experiments similar to (C) were performed except ascorbate or FeSO₄ were added to the extract at 100 μM each. (E) Nuclear HIF-1α accumulation was monitored in U251 grown for four hours in MEM supplemented with either DETA-NO (300 μM), or for four hours in glucose-free Krebs supplemented with pyruvate (3 mM) or oxaloacetate (3 mM). Cells were co-incubated with ascorbate (100 μM), Fe(II) (100 μM) or GSH (5 mM) as indicated. Decay of NO-induced HIF-1α was followed after washing with glucose-free Krebs±Fe(II) or Fe(III) supplementation (100 μM each). (F) Decay of HIF-1α induced by either pyruvate, oxaloacetate, or DFO was followed after washing with glucose-free Krebs±Fe(II) or Fe(III) supplementation (100 μM each). Unlabeled lanes indicate cells cultured in glucose-free Krebs buffer alone. (G) Intracellular iron levels were measured via fluorescence in calcein loaded U251 cells exposed to the indicated agents.

FIG. 7. Basal HIF-1 activation through glucose metabolism correlates with invasive cancer phenotype

The head and neck sqaumous cell carcinoma cell lines O22 and 22B display differential basal levels of HIF-1α (A), VEGF (B), invasiveness (C), glucose consumption (D), pyruvate production (E), and lactate accumulation (F). (G) and (H) Cellular HPH activity in 22B cell extracts was measured via ³⁵S-pVHL pull down assays. Cell extracts were prepared at indicated times following media replacement. HPH activity was measured with (G) and without (H) ascorbate addition to the assay. (I) Western blot analysis of HPH-2 and HPH-3 in U251 extracts prepared at indicated times following media replacement. (J) Ascorbate selectively lowers basal HIF-1α buildup in 22B cells. K. Pyruvate (10 mM)-enhanced HIF-1α buildup in 22B cells is readily reversed by reversed by ascorbate and Fe(II)>Fe(III) (100 μM each). Additions were made during last 30 minutes of the 24 hours incubation period. (L) Effect of ascorbate on basal expression of HIF-1 regulated genes in 22B cells. (M) Effect of ascorbate on basal invasiveness of 22B cells.

FIG. 8. Model for regulation of HIF-prolyl hydroxylases by oxygen availability (A-C) or by reversible inactivation (D-E). See specification for details

### DETAILED DESCRIPTION OF THE INVENTION

General Description

HIF-1 is known to regulate close to fifty genes that promote cell survival. A novel mechanism of HIF-1 activation by glycolytic metabolites was recently described in International Patent Application PCT/US04/37045 herein incorporated by reference in its entirety. This mode of HIF-1 activation was shown to be oxygen-independent, and mediated by specific 2-oxoacid metabolites of glucose. Applicants have now identified ascorbate, cystine, cysteine, histidine, glutathione and derivatives thereof as HIF-1 inhibitors. These agents selectively lower HIF-1 activation by the 2-oxoacid glucose metabolites but not HIF-1 activation by hypoxia. The current invention includes the use of the ascorbate, cystine, cysteine, histidine, glutathione and derivatives thereof to lower cellular activity of the transcription factor HIF-1 and treat diseases associated with excessive HIF-1 activity, such as cancer.

HIF-1 activation by glycolytic metabolites underlies the high basal level of HIF-1 activity seen in cancer cells. Agents which lower basal HIF-1 activity in cancer cells and can lower gene expression for HIF-1 regulated tumor growth factors. These agents appear to lower the high basal levels of HIF-1 in cancer cells without impairing the induction of HIF-1 by hypoxia. Some of the medical complications of hyperglycemia may manifest themselves through the glycolytic activation of HIF-1. Thus, diabetic complications such as diabetic retinopathy may also be treated by any one of ascorbate, cystine, cysteine, histidine, glutathione and/or derivatives thereof.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, the term “binding” refers to the adherence of molecules to one another, such as, but not limited to, enzymes to substrates, proteins to proteins, small molecules to proteins, transcription factor proteins to DNA, and DNA or RNA strands to their complementary strands. Binding occurs because the shape and chemical nature of parts of the molecule surfaces are complementary. A common metaphor is the “lock-and-key” used to describe how enzymes interact with their substrate.

As used herein, the term “transcription factor” refers to any protein or protein complex that binds to specific regulatory regions of DNA to stimulate gene expression.

As used herein, the term “gene expression” refers to the enhanced production of messenger RNA (mRNA) from DNA, which eventually leads to increased protein expression and to increased protein activity.

As used herein, the term “hypoxia” refers to oxygen tensions below about five (5) percent. Normal air is composed of 20 to 21 percent oxygen, a condition referred to as “normoxia” in the art.

As used herein, the term “agent” or “therapeutic agent” refers to any molecule, which binds to a HIF-1α hydroxylating enzyme and inhibits its activity. Examples include, but are not limited to, ascorbate, cysteine, histidine, glutathione, cytsteine-hystidine, hystidine-hystidine and methyl-, ethyl-, and glycerol-esters of cystine and histidine. Other example may include agents that combine the structure of cystine and histidine as in the dipeptide cystinyl-histidine.

Modulation of HIF-1α Expression

The present methods of the invention include administration of agents which modulate (e.g., inhibit) HIF-1α expression and activity to treat diseases associated with abnormal HIF-1 expression and/or activity. The agents ascorbate, cystine, cysteine, histidine, glutathione and their derivatives protect enzymes involved in HIF-1α degradation from becoming inactivated. This results in lowering the high basal levels of HIF-1α in cancer cells but does not impair the regular induction of HIF-1 by hypoxia in normal cells.

Modulation of the gene, gene fragments, or the HIF-1α protein and fragments is useful in gene therapy to treat disorders associated with the activity of this protein. In one embodiment, expression is modulated to decrease activity in diseases associated with abnormal HIF-1α protein activity (e.g., cancer). Expression vectors may be used to introduce the nucleic acids of the invention into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g., plasmid, retrovirus, lentivirus, adenovirus and the like, where the vectors are able to transiently or stably be maintained in the cells, usually for a period of at least about one day, more usually for a period of at least about several days to several weeks.

Nucleic acids encoding the HIF-1α protein or fragments thereof may be introduced into tissues or host cells by any number of routes, including viral infection, microinjection, or fusion of vesicles. Jet injection may also be used for intramuscular administration, as described by Furth et al. (1992) Anal. Biochem. 205, 365-368. The nucleic acid may be coated onto gold microparticles, and delivered intradermally by a particle bombardment device, or “gene gun” as described in the literature (see, for example, Tang et al. (1992) Nature 356, 152-154), where gold microprojectiles are coated with nucleic acids, then bombarded into skin cells.

Antisense molecules can be used to down-regulate expression of nucleic acids or proteins of the invention in cells. The anti-sense reagent may be antisense oligonucleotides, particularly synthetic antisense oligonucleotides having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g., by reducing the amount of mRNA available for translation, through activation of RNAse H or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about seven, usually at least about twelve, and more usually at least about twenty nucleotides in length. Typical antisense oligonucleotides are usually not more than about five-hundred, more usually not more than about fifty, and even more usually not more than about thirty-five nucleotides in length, where the length is governed by efficiency of inhibition, specificity, including absence of cross-reactivity, and the like. It has been found that short oligonucleotides, of from seven to eight bases in length, can be strong and selective inhibitors of gene expression (see Wagner et al. (1996) Nat. Biotech. 14, 840-844).

A specific region or regions of the endogenous sense strand mRNA sequence is chosen to be complimented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1996) Nat. Biotech. 14, 840-844). Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

As an alternative to anti-sense inhibitors, catalytic nucleic acid compounds, e.g., ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (see, for example, WO 95/23225; Beigelman et al. (1995) Nucl. Acids Res. 23, 4434-4442). Examples of oligonucleotides with catalytic activity are described in WO 95/06764.

Methods of Treatment

As discussed above, HIF-1 has been shown to play a critical role in tumor growth, including angiogenesis and glycolysis, and metastases, and identified as a potential target for anti-cancer therapeutic strategies. (Semenza (2003) Nature Rev. 3, 721-732; Williams et al. Oncogene (2002) 21, 282-90; Griffiths et al. (2002) Cancer Res. 62, 688-95; Welsh et al. (2003) Mol. Cancer. Ther. 2, 235-43). HIF-1 has been shown to be overexpressed in breast cancer and potentially associated with more aggressive tumors (Bos et al. (2001) J. Natl. Cancer Inst. 93, 309-314). In addition, HIF-1 has been recently identified as a critical link between inflammation and oncogenesis (Jung et al. (2003) FASEB J. 17, 2115-2117). HIF-1α overexpression in biopsies of brain, breast, cervical, esophageal, oropharyngeal and ovarian cancers is correlated with treatment failure and mortality. Increased HIF-1 activity promotes tumor progression, and inhibition of HIF, such as HIF-1, could represent a novel approach to cancer therapy. Therefore, the present invention comprises administration of agents for modulating (e.g., inhibiting) expression and/or activity of HIF-1α to inhibit cancer cell differentiation and proliferation. Such methods will be useful in the treatment of disorders associated with abnormal cell proliferation and differentiation, including cancer. Treatment of mammals (i.e. humans) diagnosed with diseases involving cell proliferation and differentiation include, but are not limited to, excessive vascularization (e.g., diabetic retinopathy, arteriovenous malformations, and angiomas) and are also included in the methods of the invention (see below). Administration of ascorbate, cystine, cysteine, histidine, glutathione and derivatives thereof, which inhibit HIF-1α activity, are useful for the treatment of these diseases. Thus, inhibiting HIF-1 using the methods and agents of the present invention is useful for the prevention and treatment of cancer, offering a new anti-cancer strategy, either alone or in combination with other treatment options. Inhibition of HIF-1 by administering the agents of the present invention may also enhance the efficacy of other cancer therapies, such as radiation therapy and/or treatment with chemotherapeutic agents. Specific cancer targets include, without limitation, solid tumor malignancies and Non-Hodgkin's lymphoma.

As shown in the examples below, the agents and methods of the present invention effectively inhibit tumor growth in cell-based assays and are thus promising anti-cancer agents for the treatment of a variety of tumors, including, without limitation, pancreatic, colon, and lung cancer.

In addition, HIF-1 has been identified as a target for diseases in general in which hypoxia is a major aspect, such as, for example, heart disease, stroke (Giaccia et al. (2003) Nat. Rav. Drug Discov. 2, 803-822), and chronic lung disease. Accordingly, the agents of the present invention can also be used for the prevention and treatment of hypoxia-associated diseases and pathologic conditions, such as, for example, cardiovascular diseases (including ischemic cardiovascular diseases), such as myocardial ischemia, myocardial infarction, congestive heart failure, cardiomyopathy, cardiac hypertrophy, and stroke.

The ability to down-regulate expression of multiple glycolytic enzymes and glucose transporters downstream in the HIF-1α enzyme pathway also offers applications for treating obesity. Administration of agents which inhibit HIF-1α expression and/or activity which subsequently inhibit enzymes downstream in the HIF-1α pathway is also useful for the treatment of obesity.

Agents and methods of the invention are also useful for opthalmology, including ameliorating, treating and/or preventing diabetic retinopathy, which is the leading cause of blindness in the United States. Additional ophthalmologic targets include Age-related Macular Degeneration (AMD), either wet (neovascular) or dry (non-neovascular), and corneal neovascularization associated with transplants.

Agents and methods of the invention are also useful for the prevention and treatment of pathogenic blood vessel growth, associated, for example, with psoriasis, corneal neovascularization, infection or trauma.

New blood vessel growth is also seen in the penumbral regions of ischemic lesions such as those in stroke. While such HIF-1 mediated effects attempt to restore blood supply to the lesioned area, the magnitude of this response can sometimes promote tissue injury. This is because newly formed blood vessels are leaky and can produce vaso genic edema. In the brain this can lead to increased intracranial pressure and herniation. It is envisioned that the agents and methods of this invention can be used in the management of vasogenic edema in the setting of tumors and stroke. Among the glucose metabolites that have been identified to induce HIF-1 is the branched chain keto-acids, alpha-keto isovaleric acid. This metabolite is highly elevated in a rare inherited metabolic disease known as maple syrup urine disease. Patients with this disorder sometimes present with life threatening cerebral edema. It is envisioned that the agents and methods of this invention can be used in the management of maple syrup urine disease. Another disorder linked to HIF-1 overactivation with exaggerated vascular endothelial growth factor induction is pulmonary edema, which is seen in some individuals that ascend rapidly to high altitudes. Pulmonary edema, headaches, and plycythemia, inducible by overactive HIF-1 are at the heart of another disorder known as chronic mountain sickness. It is envisioned that the agents and methods of this invention can be used in the management of chronic mountain or high altitude sickness.

Increased angiogenesis along with leukocyte, T-cell and macrophage mediated immune reactions are a key component of synovitis and bone modeling in arthritis. Preclinical studies of angiogenesis inhibitors in animals models of inflammatory arthritis support the hypothesis that inhibition of neovascularization may reduce inflammation and joint damage. Therefore, additional therapeutic targets include inflammatory diseases, including arthritis, such as rheumatoid arthritis (RA), and musculoskeletal disorders. For further details see, e.g. Walsh and Haywood (2001) Curr. Opin. Investig Drugs 2, 1054-63. In addition, similar to tumor growth, endometriotic implants require neovascularization to establish, grow and invade. This process can be prevented by the agents of the present invention thus providing a role for the agents and methods of this invention in contraception therapeutic abortion. See also, Taylor et al. (2002) Ann NY Acad. Sci. 955, 89-100. For further details of HIF, such as HIF-1 associated diseases see, e.g. Semenza (2000) Appl. Physiol. 83, 1474-1480.

To determine the dose in which the agents of the invention will be effective in the treatment, prevention or management of cancer, neovascular diseases and/or inflammatory diseases can be determined by standard research techniques. For example, the dosage of the composition which will be effective in the treatment, prevention or management of cancer, neovascular diseases and/or inflammatory diseases can be determined by administering the agents to an animal model such as, e.g., the animal models disclosed herein or known to those skilled in the art. In addition, in vitro assays may optionally be employed to help identify optimal dosage ranges.

Selection of the preferred effective dose can be determined (e.g., via clinical trials) by a skilled artisan based upon the consideration of several factors which will be known to one of ordinary skill in the art. Such factors include the disease to be treated or prevented, the symptoms involved, the patient's body mass, the patient's immune status and other factors known by the skilled artisan to reflect the accuracy of administered pharmaceutical compositions.

The precise dose to be employed in the formulation will also depend on the route of administration, and the seriousness of the disease to be treated, and should be decided according to the judgment of the practitioner and each patient's circumstances. Effective doses may be extrapolated from dose-response curves derived from in vitro or animal model test systems. For agents of the invention, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Certain preferred embodiments of the invention provides for any method of administrating lower doses of known prophylactic or therapeutic agents than previously thought to be effective for the prevention, treatment, management or amelioration of cancer, neovascular diseases and/or inflammatory diseases. Preferably, lower doses of known anti-cancer therapies are administered in combination with lower doses of the agents of the invention.

The invention also includes methods and compositions for the prevention, management or treatment of cancer, neovascular diseases and/or inflammatory diseases in a mammal, including a human, comprising administering to said mammal an amount of a cytotoxic agent, or a pharmaceutical composition comprising an amount of the cytotoxic agent, that is effective in enhancing the effect of the agents of the invention. Said cytotoxic drug can be administered prior to or simultaneously with said agents. In a preferred embodiment, said agent for modulating (e.g., inhibiting) expression and/or activity of HIF-1αinhibit cancer cell differentiation and proliferation. Such methods will be useful in the treatment of disorders associated with abnormal cell proliferation and differentiation, including cancer. Treatment of other diseases involving cell proliferation and differentiation include, but are not limited to, excessive vascularization (e.g., diabetic retinopathy, arteriovenous malformations, and angiomas) and are also included in the methods of the invention (see below). Administration of ascorbate, cystine, cysteine, histidine, glutathione and derivatives thereof, which inhibit HIF-1α activity, are useful for the treatment of these diseases. Inhibiting HIF-1 by the agents of the present invention is useful in the prevention and treatment of cancer, offering a new anti-cancer strategy, either alone or in combination with other treatment options. Inhibition of HIF-1 by administering the agents of the present invention may also enhance the efficacy of other cancer therapies, such as radiation therapy and/or treatment with chemotherapeutic agents. Specific cancer targets include, without limitation, solid tumor malignancies and Non-Hodgkin's lymphoma.

Diseases Associated with Neo-Vascularization

Angiogenesis is the process by which new blood vessels form by developing from pre-existing vessels. This multi-step process involves signaling to endothelial cells, which results in (1) dissolution of the membrane of the originating vessel, (2) migration and proliferation of the endothelial cells, and (3) formation of a new vascular tube by the migrating cells (Alberts et al. (1994) Molecular Biology of the Cell. Garland Publishing,). While this process is employed by the body in beneficial physiological events such as wound healing and myocardial infarction repair, it also exploited by unwanted cells and it causes undesirable diseases and conditions. Diseases and conditions that are associated with abnormal, excessive blood vessel development can be treated or prevented by the agents, methods and formulations of the present invention.

Examples of the diseases and conditions that can be treated or prevented include, but are not limited to, cancer (e.g., brain cancer and other solid tissue tumors), vascularization of the cornea in newborns placed in high oxygen incubators, and vascularization of the eye, skin, and other organs following injury or infection. In addition to vascularization as a result of injury or infection, other eye diseases such as neovascular eye diseases caused by uncontrolled angiogenesis can also be treated or prevented. In this instance, it is noted that pathological angiogenesis from retinal and choroidal circulations is a serious consequence of many eye diseases. Retinal neovascularization occurs in diabetic retinopathy, sickle cell retinopathy, retinal vein occlusion, and retinopathy of prematurity (ROP). Occlusion of the central retinal vein, or one of its branches, can lead to rapid diminution of vision with later sequelae of retinal neovascularization. The vascular supply to the optic nerve, derived from the choroidal system, may be interrupted in anterior ischemic optic neuropathy. New blood vessels arising from choroidal capillaries lead to choroidal neovascularization which occurs in age-related macular degeneration, either wet (neovascular) or dry (non-neovascular), and several macular diseases. Thus, the invention contemplates modulation of the HIF-1 transcription factor for the treatments of diseases where increased vascularization is a pathology of the disease. This can be accomplished by modulation of HIF-1α activity as described herein including, but not limited to, administration of ascorbate, cystine, cysteine, histidine, glutathione and derivatives thereof which inhibit HIF-1α activity and are useful for the treatment of neovascular diseases.

Inappropriate angiogenesis has also been implicated, for example, in deleterious remodeling in atherosclerosis and restenosis, idiopathic pulmonary fibrosis, acute adult respiratory distress syndrome, asthma, dermatitis, psoriasis, synovitis, osteomyelitis, arthritis, including rheumatoid arthritis, inflammatory bowel and periodontal disease. All of these diseases can be treated or prevented with the agents, methods and formulations of the present invention. Thus, in one embodiment of the invention, modulation of HIF-1α activity as described herein including, but not limited to, administration of ascorbate, cystine, cysteine, histidine, glutathione and derivatives thereof which inhibit HIF-1α activity and are useful for the treatment of inflammatory conditions. In an other embodiment, the methods of the invention encompass the administration of one or more anti-inflammatory agents, such as, but not limited to, non-steroidal anti-inflammatory drugs (NSAIDs), steroidal anti-inflammatory drugs, beta-agonists, anticholingeric agents, and methyl xanthines with agents which modulate HIF-1α activity.

Angiogenesis is required for the growth and metastasis of solid tumors. Studies have confirmed that in the absence of angiogenesis, tumors rarely have the ability to develop beyond a few millimeters in diameter (Isayeva et al. (2004) Int. J. Oncol. 25, 335-43). Angiogenesis is also necessary for metastasis formation by facilitating the entry of tumor cells into the blood circulation and providing new blood vessels that supply nutrients and oxygen for tumor growth at the metastatic site (Takeda et al. (2002) Ann Surg. Oncol. 9, 610-16). Thus, in one embodiment of the invention, modulation of HIF-1α activity as described herein including, but not limited to, administration of ascorbate, cystine, cysteine, histidine, glutathione and derivatives thereof, which inhibit HIF-1α activity and are useful for the treatment of solid tumors by inhibiting angiogenesis. For example, the invention contemplates treatment of lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma and pituitary adenoma. In another embodiment, the methods of the invention encompass the administration of at one additional anti-angiogenic agent (e.g., angiostatin or endostatin) in combination with the agents of the invention to a mammal in need thereof.

Abnormal neovascularization is also seen in various eye diseases, where it results in hemorrhage and functional disorder of the eye, contributing to the loss of vision associated with such diseases as retinopathy of prematurity, diabetic retinopathy, retinal vein occlusion, and age-related macular degeneration (Yoshida et al. (1999) Histol Histopathol. 14, 1287-94).

These conditions are the leading causes of blindness among infants, those of working age and the elderly (Aiello (1997) Ophthalmic Res. 29, 354-62). Thus, in another embodiment of the invention, modulation of HIF-1α activity as described herein including, but not limited to, administration of ascorbate, cystine, cysteine, histidine, glutathione and derivatives thereof which inhibit HIF-1α activity and are useful for the treatment of neovascular diseases of the eye, including, but not limited to retinopathy of prematurity, diabetic retinopathy, retinal vein occlusion, and age-related macular degeneration, either wet (neovascular) or dry (non-neovascular).

Abnormal neovascularization is also associated with the following diseases: vascular malformations, artherosclerosis, transplant arteriopathy, obesity (angiogenesis induced by a fatty diet), warts, pyogenic granulomas, primary pulmonary hypertension, nasal polyps, endometriosis, uterine bleeding, ovarian cysts and ovarian hyperstimulation. Thus, in another embodiment of the invention, modulation of HIF-1 or, activity as described herein including, but not limited to, administration of ascorbate, cystine, cysteine, histidine, glutathione and derivatives thereof which inhibit HIF-1α activity and are useful for the treatment of the above identified diseases including any disease in which neovascularization is a pathology of the disease.

Vascular endothelial growth factor (VEGF) is a particularly potent angiogenic factor that acts as an endothelial cell-specific mitogen during angiogenesis (Binetruy-Tourniere et al. (2000) EMBO J. 19, 1525-33). VEGF has been implicated in promoting solid tumor growth and metastasis by stimulating tumor-associated angiogenesis (Lu et al. (2003) J. Biol. Chem. 278, 43496-43507). VEGF has been found in the synovial fluid and serum of patients with rheumatoid arthritis (RA), and its expression is correlated with disease severity (Clavel et al. (2003) Joint Bone Spine. 70, 321-326). VEGF has also been implicated as a major mediator of intraocular neovascularization and permeability. Transgenic mice overexpressing VEGF demonstrate clinical intraretinal and subretinal neovascularization, and form leaky intraocular blood vessels detectable by angiography, demonstrating their similarity to human disease (Miller (1997) Am. J. Pathol. 151, 13-23). Activation of the HIF-1 transcription factor has been linked to increased VEGF activity (Ryan et al. (2000) Cancer Res. 60, 4010-4015). Thus, the invention also contemplates regulation of VEGF activity by modulation of HIF-1α activity as described herein including, but not limited to, administration of ascorbate, cystine, cysteine, histidine, glutathione and derivatives thereof which inhibit HIF-1α activity and are useful for the regulation of angiogenesis. In another embodiment, the methods of the invention encompass the administration of one or anti-VEGF agents in combination with the agents of the invention to a mammal in need thereof.

Cancer Therapy

A neoplasm, or tumor, is a neoplastic mass resulting from abnormal uncontrolled cell growth which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell (1976) Basic Pathology, 2d Ed., W. B. Saunders, pp. 68-122). Cancer can arise in many sites of the body and behave differently depending upon its origin. Cancerous cells destroy the part of the body in which they originate and then spread to other part(s) of the body where they start new growth and cause more destruction.

Currently, cancer therapy may involve surgery, chemotherapy, hormonal therapy and/or radiation treatment to eradicate neoplastic cells in a patient (see, for example, Stockdale (1998) Principles of Cancer Patient Management, in Scientific American: Medicine, vol. 3, Rubenstein and Federman, eds., Chapter 12, Section IV). Recently, cancer therapy could also involve biological therapy or immunotherapy. All of these approaches pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and although can be effective, is often used to prevent or delay recurrence of cancer after other treatments have removed the majority of the cancer cells. Biological therapies/immunotherapies are limited in number and may produce side effects such as rashes or swellings, flu-like symptoms, including fever, chills and fatigue, digestive tract problems or allergic reactions.

With respect to chemotherapy, there is a variety of chemotherapeutic agents available for treatment of cancer. A significant majority of cancer chemotherapeutics act by inhibiting DNA synthesis, either directly, or indirectly by inhibiting the biosynthesis of the deoxyribonucleotide triphosphate precursors, to prevent DNA replication and concomitant cell division (see, for example, Gilman et al. (1990) Goodman and Gilman's: The Pharmacological Basis of Therapeutics, Eighth Ed. Pergamom Press, New York). These agents, which include alkylating agents, such as nitrosourea, anti-metabolites, such as methotrexate and hydroxyurea, and other agents, such as etoposides, campathecins, bleomycin, doxorubicin, daunorubicin, etc., although not necessarily cell cycle specific, kill cells during S phase because of their effect on DNA replication. Other agents, specifically colchicine and the vinca alkaloids, such as vinblastine and vincristine, interfere with microtubule assembly resulting in mitotic arrest. Chemotherapy protocols generally involve administration of a combination of chemotherapeutic agents to increase the efficacy of treatment.

Despite the availability of a variety of chemotherapeutic agents, chemotherapy has many drawbacks (see, for example, Stockdale (1998) Principles of Cancer Patient Management, in Scientific American Medicine, vol. 3, Rubenstein and Federman, eds., Chapter 12, Section X). Almost all chemotherapeutic agents are toxic, and chemotherapy causes significant, and often dangerous, side effects, including severe nausea, bone marrow depression, immunosuppression, etc. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even those agents that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

There is a significant need for alternative cancer treatments, particularly for treatment of cancer that has proved refractory to standard cancer treatments, such as surgery, radiation therapy, chemotherapy, and hormonal therapy. Further, it is uncommon for cancer to be treated by only one method. Thus, there is a need for development of new therapeutic agents for the treatment of cancer and new, more effective, therapy combinations for the treatment of cancer.

Cancer is aggravated by elevated HIF-1 levels. This is because HIF-1 activation promotes the survival, metabolism, invasiveness, and neovascularization of cancer cells. The inventors have shown (see below) that lowering the high basal levels of HIF-1α in cancer cells impairs survival and invasiveness of these cells. Thus, the present invention, therefore, contemplates methods of administration of agents for modulating (e.g., inhibiting) expression and/or activity of HIF-1α to inhibit cancer cell differentiation and proliferation to a person in need thereof. Such methods will be useful in the treatment of disorders associated with abnormal cell proliferation and differentiation, including cancer. Thus, in one embodiment of the invention, cancer or one or more symptoms thereof, is prevented, treated, managed or ameliorated by the administration of agents which modulate HIF-1α activity as described herein including, but not limited to administration of ascorbate, cystine, cysteine, histidine, glutathione and derivatives thereof which inhibit HIF-1α. In another embodiment, combination therapies comprising agents which modulate HIF-1α activity and one or more therapies such as, but not limited to, chemotherapies, radiation therapies, hormonal therapies, and/or biological therapies/immunotherapies are also contemplated.

In a specific embodiment, the methods of the invention encompass the administration of one or more angiogenesis antagonists with agents which modulate HIF-1α activity. In another embodiment, the methods of the invention encompass the administration of one or more immunomodulatory agents, such as but not limited to, chemotherapeutic agents and non-chemotherapeutic immunomodulatory agents with agents which modulate HIF-1α activity.

Specific examples of anti-cancer agents that can be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin Vitaxin; vorozole; zanoterone; zeniplatin; zilascorb; and zinostatin stimalamer.

In more particular embodiments, the present invention also comprises the administration of therapeutic agents which modulate HIF-1α activity as described herein including, but not limited to administration of ascorbate, cystine, cysteine, histidine, glutathione and derivatives thereof which inhibit HIF-1α activity in combination with the administration of one or more therapies such as, but not limited to, anti-cancer agents. Cancer treatments include the treatment of lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma and pituitary adenoma. When used in a combination therapy, the dosages and/or the frequency of administration listed in may be decreased.

Autoimmune and Inflammatory Diseases

Inflammation is characterized by localized hypoxia caused by increased metabolic demand. It has become apparent in recent years that the heterodimeric transcription factor HIF-1 is key to the molecular mechanisms by which hypoxia influences changes in cellular physiology. Genes induced by HIF-1 include those necessary for cellular, whole-tissue, and whole-animal adaptive responses to hypoxia. Recently has it become apparent that HIF-1 may control gene expression in more diverse settings, such as inflammation (Cramer et al. (2003) Cell Cyle, 2, 192-193). Studies reveal that, as a transcriptional regulator of O₂ integrin expression, HIF-1 may function to control the migration of myeloid leukocytes to inflammatory lesions.

As discussed above, HIF-1 expression depends predominantly on the degradation of its α-subunit in normoxia through the ubiquitin-proteasomal pathway. The initiating step in this process is iron-dependent proline hydroxylation within the oxygen-dependent degradation domain. Recent studies have revealed a potentially central role for HIF-1 in endogenous protective pathways within a variety of inflammatory diseases, including respiratory distress syndrome, retinitis, diabetes, and arthritis (Semenza et al. (2000) Adv. Exp. Med. Biol. 475, 123-130). In addition, a recent cDNA profiling study suggested that HIF-1α expression is induced in ulcerative colitis (Lawence et al. (2001) 10, 445-456).

It is now apparant that leukocyte responses to hypoxia contribute to inflammation and autoimmune disease progression. In both acute conditions, such as ischemia-reperfusion injury, and chronic inflammation, such as occurs in arthritis, it is well documented that leukocytes are exposed to hypoxia (Lewis et al. (1999) J. Leukocyte Biol. 66, 889-900). In addition, there is evidence to suggest that hypoxia may result in the increased expression of molecules, like the β₂ integrins, that mediate leukocyte adhesion and effect leukocyte extravasation, chemotaxis, and phagocytosis (Kong et al. (2004) Proc. Natl. Acad. Sci. USA 101, 10440-10445). Also, there is evidence to suggest that immunomodulatory peptides, including IL-1 and TNFα, stimulate HIF-1 dependent gene expression even in normoxic cells (Hellwig-Burgel et al. (2005) J. Interferon Cytokine Res. 25, 297-310). Therefore, chronic inflammation mediated by leukocytes such as T-cells, macrophages and/or neutrophils require HIF-1 activation in order to produce an inflammatory response. Thus, the invention contemplates the modulation of the HIF-1 transcription factor for the treatments of diseases where leukocytes such as macrophages, T-cells and neutrophils are activated and cause inflammation as a pathology of the disease. This can be accomplished by modulation of HIF-1α activity as described herein including, but not limited to, administration of ascorbate, cystine, cysteine, histidine, glutathione and derivatives thereof which inhibit HIF-1α activity and are useful for the treatment of inflammatory diseases.

The compositions and methods of the invention described herein are useful for the prevention or treatment of autoimmune disorders and/or inflammatory disorders. Examples of autoimmune disorders include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome (Moersch-Woltmann syndrome), systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis.

Examples of inflammatory disorders include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections. The compositions and methods of the invention can be used with one or more conventional therapies that are used to prevent, manage or treat the above diseases.

The compositions and methods described herein are particularly useful for the prevention or treatment of rheumatoid arthritis, spondyloarthropathies (e.g., psoriatic arthritis, ankylosing spondylitis, Reiter's Syndrome (a.k.a., reactive arthritis), inflammatory bowel disease associated arthritis, and undifferentitated spondyloarthropathy), psoriasis, undifferentiated arthropathy, and arthritis. The compositions and methods described herein can also be applied to the prevention, treatment, management or amelioration of one or more symptoms associated with inflammatory osteolysis, other disorders characterized by abnormal bone reabsorption, or disorder characterized by bone loss (e.g., osteoporosis).

Pharmaceutical Formulations

For pharmaceutical uses, the agents of the present invention may be used in combination with a pharmaceutically acceptable carrier, and can optionally include a pharmaceutically acceptable diluent or excipient. The present invention thus also provides pharmaceutical compositions suitable for administration to a subject. The carrier can be a liquid, so that the composition is adapted for parenteral administration, or can be solid, i.e., a tablet or pill formulated for oral administration. Further, the carrier can be in the form of a nebulizable liquid or solid so that the composition is adapted for inhalation. When administered parenterally, the composition should be pyrogen free and in an acceptable parenteral carrier. Active agents can alternatively be formulated or encapsulated in liposomes, using known methods.

The pharmaceutical compositions of the invention comprise an effective amount of modulate HIF-1α activity as described herein including, but not limited to administration of ascorbate, cystine, cysteine, histidine, glutathione and derivatives thereof in combination with the pharmaceutically acceptable carrier. The compositions may further comprise other known drugs suitable for the treatment of the particular disease being targeted. An effective amount of the agent of the present invention is that amount that blocks, inhibits or reduces stimulation of endothelial cells compared to that which would occur in the absence of the agent; in other words, an amount that decreases the angiogenic activity of the endothelium, compared to that which would occur in the absence of the agent. The effective amount (and the manner of administration) will be determined on an individual basis and will be based on the specific therapeutic molecule being used and a consideration of the subject (size, age, general health), the condition being treated (cancer, arthritis, eye disease, etc.), the severity of the symptoms to be treated, the result sought, the specific carrier or pharmaceutical formulation being used, the route of administration, and other factors as would be apparent to those skilled in the art. The effective amount can be determined by one of ordinary skill in the art using techniques as are known in the art. Therapeutically effective amounts of the agents described herein can be determined using in vitro tests, animal models or other dose-response studies, as are known in the art.

The agents of the present invention can be administered acutely (i.e., during the onset or shortly after events leading to inflammation), or can be administered during the course of a degenerative disease to reduce or ameliorate the progression of symptoms that would otherwise occur. The timing and interval of administration is varied according to the subject's symptoms, and can be administered at an interval of several hours to several days, over a time course of hours, days, weeks or longer, as would be determined by one skilled in the art. A typical daily regime can be from about 0.01 μg/kg body weight per day, from about 1 mg/kg body weight per day, from about 10 mg/kg body weight per day, from about 100 mg/kg body weight per day.

The agents of the invention may be administered by intravenously, orally, intranasally, intraocularly, intramuscularly, intrathecally, or by any suitable route in view of the agent's formulation and the disease to be treated. Agents for the treatment of inflammatory arthritis can be injected directly into the synovial fluid. Agents for the treatment of solid tumors may be injected directly into the tumor. Agents for the treatment of skin diseases may be applied topically, for instance in the form of a lotion or spray. Intrathecal administration, i.e. for the treatment of brain tumors, can comprise injection directly into the brain. Alternatively, agents may be coupled or conjugated to a second molecule (a “carrier”), which is a peptide or non-proteinaceous moiety selected for its ability to penetrate the blood-brain barrier and transport the active agent across the blood-brain barrier. Examples of suitable carriers are disclosed in U.S. Pat. Nos. 4,902,505; 5,604,198; and 5,017,566, which are herein incorporated by reference in their entirety.

Methods to Identify HIF-1α Binding Partners

Another embodiment of the present invention provides methods for use in isolating and identifying binding partners of HIF-1α. In general, HIF-1α protein is mixed with a potential binding partner or an extract or fraction of a cell under conditions that allow the association of potential binding partners with HIF-1α protein. After mixing, peptides, polypeptides, proteins or other agents (e.g., cyteine or histidine) that have become associated with HIF are then separated from the mixture. The binding partner that bound to HIF can then be removed and further analyzed. To identify and isolate a binding partner, the HIF-1α entire protein can be used. Alternatively, a fragment of the protein can be used.

As used herein, a cellular extract refers to a preparation or fraction that is made from a lysed or disrupted cell. The preferred source of cellular extracts will be cells derived from human skin tissue or the human respiratory tract or cells derived from a biopsy sample of human lung tissue in patients with allergic hypersensitivity. Alternatively, cellular extracts may be prepared from normal tissue or available cell lines, particularly cancer cell lines, including glioma cell lines.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the agents of the invention under conditions in which association of the HIF-1α protein with the binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density/sediment centrifugation can be used.

After removal of non-associated cellular constituents found in the extract, the binding partner can be dissociated from the complex using conventional methods. For example, dissociation can be accomplished by altering the salt concentration or pH of the mixture. To aid in separating associated binding partner pairs from the mixed extract, the agent of the invention can be immobilized on a solid support. For example, the agent can be attached to a nitrocellulose matrix or acrylic beads. Attachment of the agent to a solid support aids in separating binding partners from other constituents found in the extract. The identified binding partners can be either a single protein or a complex made up of two or more proteins. Alternatively, binding partners may be identified using a Far-Western assay according to the procedures of Takayama et al. (1997) Methods Mol. Biol. 69, 171-184 or Sander et al. (1996) J. Gen. Virol. 77, 991-996 or identified through the use of epitope tagged proteins or GST fusion proteins.

Alternatively, the nucleic acid molecules encoding HIF-1 can be used in a yeast two-hybrid system. The yeast two-hybrid system has been used to identify other protein partner pairs and can readily be adapted to employ the nucleic acid molecules herein described.

Methods to Identify Agents that Modulate HIF-1α Expression

A novel target mechanism for cancer drug development has been identified. The agents ascorbate, cystine, cysteine, histidine, glutathione and their derivatives protect enzymes involved in HIF-1α degradation from becoming inactivated. This results in lowering the high basal levels of HIF-1α in cancer cells but does not impair the regular induction of HIF-1α by hypoxia in normal cells. The invention therefore encompasses methods of identifying agents with the same effect on HIF-1α expression as ascorbate, cystine, cysteine, histidine and glutathione.

In one embodiment of the present invention, methods are provided for identifying agents that promote HIF-1α decay. A novel mechanism for HIF-1 activation has been identified in which glucose derived metabolites cause reversible inactivation of the enzymes that hydroxylate and thus tag HIF-1α for proteolytic degradation. As with hypoxia, cells treated in culture with glucose metabolites such as pyruvate, oxaloacetate and alpha-keto isocaproate show accumulation of HIF-1α protein. However, while re-introduction of oxygen to hypoxic cells quickly promotes resumption of HIF-1 decay, this decay is far more sluggish following removal of the glucose metabolites by washing. Addition of agents such as ascorbate, cysteine, histidine, glutathione and derivatives thereof, can rapidly promote HIF-1 decay by reactivating HIF-1 hydroxylases. This mode of analysis can thus identify new agents capable of re-activating HIF-1 hydroxylases following their inactivation.

In another embodiment of the present invention, methods are provided for identifying agents that modulate the expression of a nucleic acid encoding a HIF-1α protein. Such assays may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell. Examples of agents which down-regulate the expression of HIF-1α protein include, but are not limited to, ascorbate, cystine, cysteine, histidine, glutathione and derivatives thereof.

In one assay format, cell lines that contain reporter gene fusions between the open reading frame of the HIF-1α regulated gene, or the 5′ and/or 3′ regulatory elements and any assayable fusion partner may be prepared. Numerous assayable fusion partners are known and readily available including the firefly luciferase gene and the gene encoding chloramphenicol acetyltransferase (Alam et al. (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents that modulate the expression of a nucleic acid encoding a HIF-1α protein.

Additional assay formats may be used to monitor the ability of the agent to modulate the expression of a nucleic acid encoding a HIF-1α protein. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al. (2001) Molecular Cloning—A Laboratory Manual, Cold Spring Harbor Laboratory Press).

Probes to detect differences in RNA expression levels between cells exposed to the agent and control cells may be prepared from the nucleic acids encoding a HIF-1α protein. It is preferable, but not necessary, to design probes which specifically hybridize only with target nucleic acids under conditions of high stringency. Only highly complementary nucleic acid hybrids form under conditions of high stringency. Accordingly, the stringency of the assay conditions determines the amount of complementation that should exist between two nucleic acid strands in order to form a hybrid. Stringency should be chosen to maximize the difference in stability between the probe:target hybrid and probe:non-target hybrids.

Probes may be designed from the nucleic acids encoding a HIF-1α regulated protein through methods known in the art. For instance, the G+C content of the probe and the probe length can affect probe binding to its target sequence. Methods to optimize probe specificity are commonly available in Sambrook et al. (2001) Molecular Cloning—A Laboratory Manual, Cold Spring Harbor Laboratory Press or Ausubel et al. (1995) Current Protocols in Molecular Biology, Greene Publishing.

Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The glass wafer can then be exposed to total cellular RNA or polyA RNA from a sample under conditions in which the affixed sequences will specifically hybridize. Such solid supports and hybridization methods are widely available, for example, those disclosed in WO 95/11755. By examining for the ability of a given probe to specifically hybridize to an RNA sample from an untreated cell population and from a cell population exposed to the agent, agents which up or down regulate the expression of a nucleic acid encoding the HIF-1α protein are identified.

Hybridization for qualitative and quantitative analysis of mRNA may also be carried out by using a RNase Protection Assay (i.e., RPA, see Ma et al. (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes (pH 6.4), 0.4 M NaCl and 1 mM EDTA. The resulting hybrids are then digested in a buffer comprising 40 μg/ml ribonuclease A and 2 μg/ml ribonuclease H. After deactivation and extraction of extraneous proteins, the samples are loaded onto urea/polyacrylamide gels for analysis.

In another assay format, cells or cell lines (e.g., U87 glioma) are first identified which express HIF-1α gene products physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or other detectable marker. Such a process is well known in the art (see Sambrook et al. (2001) Molecular Cloning—A Laboratory Manual, Cold Spring Harbor Laboratory Press).

Cells or cell lines transduced or transfected as outlined above are then contacted with agents (e.g., ascorbate, cystine, cysteine, histidine, glutathione and derivatives thereof) under appropriate conditions. For example, the agent in a pharmaceutically acceptable excipient is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient or control agents (ascorbate, cystine, cysteine, histidine, glutathione and derivatives thereof) is contacted with the cells and an increase or decrease in the immunologically generated signal from the agent-contacted sample compared to the control will be used to distinguish the effectiveness of the agent.

Methods to Identify Agents that Modulate Activity

The present invention provides methods for identifying agents that modulate at least one activity of the HIF-1α protein. Such methods or assays may utilize any means of monitoring or detecting the desired activity.

In one format, the specific activity of the HIF-1α protein, normalized to a standard unit, between a cell population that has been exposed to the agent to be tested compared to an un-exposed control cell population may be assayed. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes can be prepared by immunizing suitable mammalian hosts utilizing appropriate immunization protocols using the proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. may be desirable to provide accessibility to the hapten. The hapten peptides can be extended at either the amino or the carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) Biotechnology 24, 524-526 or modifications which effect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies may be recovered from the culture supernatant or from the ascites supernatant. Fragments of the monoclonal antibodies or the polyclonal antisera that contain the immunologically significant portion can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin.

Antibody regions that bind specifically to the desired regions of the protein can also be produced in the context of chimeras with multiple species origin, for instance, humanized antibodies. The antibody can therefore be a humanized antibody or human a antibody, as described in U.S. Pat. No. 5,585,089 or Riechmann et al. (1988) Nature 332, 323-327.

Agents that are assayed in the above method can be randomly selected or rationally selected or designed. As used herein, an agent is said to be randomly selected when the agent is chosen randomly without considering the specific sequences involved in the association of the HIF-1α protein alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. Examples of rationally selected agents include, but are not limited to, ascorbate, cystine, cysteine, histidine, glutathione and derivatives thereof.

The agents to be screened in the methods of the present invention can be, as examples, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention is antibodies or fragments thereof that bind to the HIF-1α protein. Antibody agents can be obtained by immunization of suitable mammalian subjects with peptides, containing as antigenic regions, those portions of the protein intended to be targeted by the antibodies.

In yet another class of agents, the present invention includes peptide mimetics that mimic the three-dimensional structure of the HIF-1α protein. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others.

In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

In another form, peptide analogs are commonly used in the pharmaceutical industry as non-peptide drugs with properties analogous to those of the template peptide. These types of non-peptide compounds are also referred to as peptide mimetics or peptidomimetics (Fauchere (1986) Adv. Drug Res. 15, 29-69; Veber & Freidinger (1985) Trends Neurosci. 8, 392-396; Evans et al. (1987) J. Med. Chem. 30, 1229-1239 which are incorporated herein by reference) and are usually developed with the aid of computerized molecular modeling.

Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage by methods known in the art.

Labeling of peptide mimetics usually involves covalent attachment of one or more labels, directly or through a spacer (e.g., an amide group), to non-interfering positions on the peptide mimetic that are predicted by quantitative structure-activity data and molecular modeling. Such non-interfering positions generally are positions that do not form direct contacts with the macromolecules to which the peptide mimetic binds to produce the therapeutic effect. Derivitization (e.g., labeling) of peptide mimetics should not substantially interfere with the desired biological or pharmacological activity of the peptide mimetic.

The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al. (1991) Proc. Natl. Acad. Sci. USA 88, 9578-9582) and using the phage display method. The two hybrid method detects protein-protein interactions in yeast (Fields et al. (1989) Nature 340, 245-246). The phage display method detects the interaction between an immobilized protein and a protein that is expressed on the surface of phages such as lambda and M13 (Amberg et al. (1993) Strategies 6, 2-4; Hogrefe et al. (1993) Gene 128, 119-126). These methods allow positive and negative selection for protein-protein interactions and the identification of the sequences that determine these interactions.

As used herein, “agents of the invention” contemplates compounds which act in a similar way as ascorbate, cysteine, histidine and glutathione. As described below, a novel mechanism for HIF-1 activation has been identified in which glucose derived metabolites cause reversible inactivation of the enzymes that hydroxylate and thus tag HIF-1α for proteolytic degradation. As with hypoxia, cells treated in culture with glucose metabolites such as pyruvate, oxaloacetate and alpha-keto isocaproate show accumulation of HIF-1α protein. However, while re-introduction of oxygen to hypoxic cells quickly promotes resumption of HIF-1 decay, this decay is far more sluggish following removal of the glucose metabolites by washing. Addition of agents such as ascorbate, cysteine, histidine, glutathione and derivatives thereof, can rapidly promote HIF-1 decay by reactivating HIF-1 hydroxylases. This mode of analysis can therefore identify completely new agents capable of re-activating HIF-1 hydroxylases following their inactivation. Thus, the invention contemplates “agents” which act in a similar fashion as ascorbate, cysteine, histidine and glutathione (the D isoforms of the amino acids).

In addition, derivatives of such agents are also contemplated. For example, dipeptides such as cytsteine-hystidine, hystidine-hystidine, cysteine-cysteine are contemplated. In addition, it is also contemplated that peptides which consists of cysteine and/or histidine in a random or recognizable pattern are also contemplated. The peptide may range from three amino acids to 100 amino acids. This peptide will promote HIF-1 decay by reactivating HIF-1 hydroxylases.

It is also contemplated that the amino acids, dipeptides and peptides of the invention can be modified in order to increase stability and/or absorption in-vivo and/or in-vitro. For example, an N-acetyl group can be added to an amino acid to increase stability. Also, methyl groups and/or ester groups can be added to peptides to increase stability and/or absorption into the cell. It is also contemplated that molecules, which mimic the effects of ascorbate, cysteine, histidine and glutathione according to the invention are also encompassed. Such molecules will have a similar effect on HIF-1α as does ascorbate, cysteine, histidine and glutathione. Such compounds can be identified using the methods described herein.

### EXAMPLES

The following working examples specifically point out preferred embodiments of the present invention, and are not to be construed as limiting in any way the remainder of the disclosure. Other generic configurations will be apparent to one skilled in the art. All cited patents, patent applications and publications referred to in this application are herein incorporated by reference in their entirety. The following materials and methods apply to all of the Examples.

Cell Culture and Hypoxia Treatment

Human U87, U251, U251-HRE (20) and U373 glioma cells were cultured in Eagle's MEM medium (Mediatech). DU145 human prostate cancer cells and 022 human head and neck cancer cells were cultured in RPMI 1640 medium (Sigma). 22B Human head and neck cancer cells were cultured in high glucose DMEM (Gibco) containing 2 mM glutamine. C6 ODD-GFP rat glioma cells were cultured in high glucose DMEM with 1.5 mg/ml G418. All culture media were supplemented with 10% fetal bovine serum and 1% (v/v) penicillin/streptomycin. Rat C6 glioma cells were cultured in high glucose DMEM. For cell hypoxia treatment, the culture dishes were sealed in a modular incubator chamber, flushed with gas containing 1% O₂, 5% CO₂ and 94% N₂ for 5 minutes, and incubated in this environment at 37° C. for indicated times.

Antibodies and Chemical Reagents

Protein extraction, western blot analysis, and immunocytochemistry were performed as previously described. Mouse monoclonal anti-HIF-1α antibodies were 610958 (BD Biosciences), and NB100-123 (Novus). Mouse monoclonal anti-GFP antibody was 1814460 (Roche). Mouse monoclonal anti-β-actin antibody was ab6276-100 (Abcam). Dimethyloxalylglycine was D1070 (Frontier Scientific). All other chemicals were from Sigma. Antibodies recognizing HPH homologues were from Novus biologicals and the antisera recognizing hydroxyproline 654 of HIF-1α was kindly provided by R. Freeman.

In Vitro Translation and HPH Prolyl Hydroxylation Assay

Human HPH-1,2,3 and VHL pcDNA3.1/V5-HIS vectors (kindly provided by R. Bruick) were used for TNT Coupled Reticulocyte Lysate in vitro transcription/translation (Promega). Cytoplasmic extract for HPH activity was made by lysing cell pellets in assay buffer (20 mM Tris, pH 7.5, 5 mM KCl, 1.5 mM MgCl₂, and 1 mM DTT) at 4° C. using a homogenizer, centrifuging at 20,000 g for 15 minutes and collecting supernatant for the assay. HPH enzymatic reactions were performed as previously described using either IVT products or cell extracts. This assay, chosen for its specificity for the HPH reaction, was linear over 30 minutes. For each reaction, either 7.5 μL of IVT products or 100 μg of cell extract protein were used as source of the enzyme. Activity was measured by either quantitative scintillation counting or by autoradiography after gel electrophoresis on a 10-20% Tris Glycine gradient gel (Invitrogen).

2-Oxoglutarate: HPH Binding Assay

Epoxy-Activated Sepharose (Amersham) coupled to 2-oxoglutarate was produced using the recommended manufacturer's protocol. 25,000 cpm of ³⁵S-labeled human HPH protein was added to 100 μL of 2-oxoglutarate sepharose gel. Gel was washed four times with the indicated buffer. Analysis of HPH binding was measured by scintillation counting.

VHL: ODD Binding Assay

35,000 cpm of ³⁵S-labeled human VHL protein was added to 1 μg of hydroxylated HIF peptide bound to Ultralink ImmunoPure Immobilized Streptavidin beads (Pierce) in the indicated buffer. The beads were washed three times with cold NTEN buffer and bound ³⁵S-VHL was measured by scintillation counting.

HIF-1 Reporter Assays

HIF-1 luciferase reporter assay was performed using U251-HRE cells as previously described (Rapisarda et al. (2002) Cancer Res. 62, 4316-4324).

RT-PCR and Quantitative RT-PCR Analysis

Total RNA was isolated using the RNeasy kit (Qiagen). For RT-PCR, 1 μg of total RNA was used with the SUPERSCRIPT One-Step System (Invitrogen). VEGF, GLUT3, and β-Actin primers were described previously. For quantitative PCR analysis, the iQ SY BR Green Supermix and MyiQ Single-Color

Real-Time PCR Detection System (Bio-Rad) was used. Total RNA (5 μg) was reverse transcribed using the High Capacity cDNA Archive Kit (Applied Biosystems). The following primers were used:

rat HO-1 (GenBank accession NM_012580):

| | |
|---|---|
| forward: | |
| 5′-AAGAGGCTAAGACCGCCTTC-3′ | (SEQ ID NO. 1) |
| reverse: | |
| 5′-CCTCTGGCGAAGAAACTCTG-3′; | (SEQ ID NO. 2) |

human CAIX (GenBank accession NM_001216):

| | |
|---|---|
| forward: | |
| 5′-CACTCCTGCCCTCTGACTTC-3′ | (SEQ ID NO. 3) |
| reverse: | |
| 5′-AGAGGGTGTGGAGCTGCTTA-3′; | (SEQ ID NO. 4) |

human GLUT3 (GenBank accession NM_006931)

| | |
|---|---|
| forward: | |
| 5′-TGACGATACCGGAGCCAATG-3′ | (SEQ ID NO. 5) |
| reverse: | |
| 5′-TCAAAGGACTTGCCCAGTTT-3′; | (SEQ ID NO. 6) |

human MMP-2 (GenBank accession NM_004530)

| | |
|---|---|
| forward = | |
| 5′-GTGGATGCCGCCTTTAACT-3′ | (SEQ ID NO. 7) |
| reverse = | |
| 5′-GGAAAGCCAGGATCCATTTT-3′. | (SEQ ID NO. 8) |

Primer temperature, concentration, and cDNA dilution optimizations were conducted before analysis and specific single-band amplification was verified through multicomponent analysis and sequencing of amplified products.
Cell Invasion Assay

Cell invasion experiments were performed using 24-well Biocoat Matrigel. Invasion Chambers with an 8 μm pore polycarbonate filter according to manufacturer's instructions (Beckton Dickinson Labware). Cells in the growing phase were trypsinized and resuspended at a concentration of 1×10⁵ cells/ml in media with 0.5% Fetal Bovine Serum (FBS). The lower compartment of the plates received 750 μl of serum free or serum media. All drug or serum treatments were added to the lower compartment of the plate prior to cell plating. 5×10⁴ cells were plated in each insert and allowed to invade for 48 hours at 37° C. in a humidified incubator with 21% O₂. Cells that remained inside the insert after 48 hours were thoroughly wiped with a cotton swab and invading cells were fixed and stained using Diff-Quick Stain Solution (Dade Begring). Stained invading cells were quantified by counting in 10 predetermined fields at 20× magnification. All treatment groups were studied in triplicate. Difference in invasion was statistically analyzed using a two-tailed student's t test.

Cellular Reactive Oxygen Species and Nitrite Measurements

Cells (80% confluent) were loaded with 5 μM CM-H2DCFDA (Molecular Probes) in Krebs buffer for one hour and then treated with 2 mM pyruvate or oxaloacetate for two hours. Positive controls were spiked twice with 40 μM H₂O₂. Cells were then collected, washed twice with PBS, and resuspended in PBS/0.1% (w/v) BSA before measuring fluorescence in a Coulter flow cytometer. Nitrite concentration in culture medium was measured as previously described.

VEGF ELISA, Glucose Metabolites, and Cellular ATP Assays

Culture medium VEGF was measured via ELISA (Quantikine). Glucose, lactate, and pyruvate were measured in phenol-red free DMEM using a CMA 600/Microdialysis Analyzer (CMA Microdialysis AB). Cellular ATP levels were measured using the ATP Bioluminescence Assay Kit CLS II (Roche).

Calcein Fluorescence Assay for Cellular Free Iron

The ability of 2-oxoacids to chelate the labile intracellular iron pool was measured using the calcein fluorescence assay as previously described. The iron chelators desferrioxamine and bipyridyl were used as positive controls.

EF-5 Assay for Cellular Hypoxia

Cellular hypoxia was monitored via immunofluorescence detection of EF-5 cellular adducts as previously described (Koch et al. (1995) Br. J. Cancer 72, 869-874).

### Example 1

### Specific Metabolic Fuels Support Hypoxia-Independent HIF-1α Levels

Serum is a major source of growth factors and the main source of iron in cell culture, while media provides metabolic fuels. When basal HIF-1α was examined levels in several cancer cell lines cultured in media with widely varying glucose concentrations but identical serum supplementation, it was observed that basal HIF-1α accumulation, which is known to be ascorbate-reversible, was typically higher in cell lines grown in high glucose DMEM (25 mM) than in RPMI (11.1 mM) or MEM (5.5 mM) (FIG. 2A). The time-dependent accumulation of basal HIF-1α in 22B cells required glucose but was not affected by the presence or absence of 10% FBS (FIG. 2B). These cells are cultured in DMEM along with the non-glycolytic energy source glutamine. With or without glucose, the 22B cells were still able to prominently induce HIF-1 under hypoxia (FIG. 2B). This not only showed that the cells could maintain prolonged viability in the presence of glutamine as the sole energy source, but also clearly distinguished glycolytic and hypoxic regulation of HIF-1 as separate mechanisms.

The U87 and U251 human glioblastoma cells (cultured in MEM) were used to further explore the relationship between glucose metabolism and basal HIF-1α. When switched from their native MEM media to Krebs buffer for 4 hours, U251 cells showed no basal HIF-1α accumulation if glucose was replaced by the non-glycolytic energy source glutamine (FIG. 2C). Despite the presence of 10 mM glutamine, basal HIF-1α accumulation in U251 cells was dose-dependently increased by glucose.

### Example 2

### 2-oxoacid Functional Groups Distinguish Endogenous HIF-1α Stabilizing Metabolites

Since glucose feeds into many metabolic pathways, it was determined which cellular intermediary metabolites were capable of promoting HIF-1α accumulation when substituted for glucose in Krebs buffer. An extensive analysis of all glycolytic, all tricarboxylic acid (TCA) cycle, and many amino acid metabolic intermediates revealed that most metabolic substrate substitutions did not significantly alter cellular ATP levels over four hours of cell culture. Moreover, only a select group of 2-oxoacids was found to promote HIF-1α accumulation. Of these, only pyruvate and oxaloacetate were found to be active in all cancer cell lines used in this study. The threshold dose for inducing HIF-1α accumulation by these 2-oxoacids over a four hours period of cell culture was determined to be about 300 μM. However, doses as low as 100 μM induced HIF-1α with culture times longer than 8 h. The branched chain 2-oxoacids α-ketoisocaproate, and α-keto-β-methylvalerate were also active in some but not all cell lines, and α-ketoisovalerate showed minor activity (FIG. 2C). The 1-carboxylate and 2-oxo functional groups common to 2-OG and to the artificial HIF-1α inducers NOG and DNOG (FIG. 2D) were also features of naturally occurring metabolic intermediates that induced HIF-1α. Succinate, fumarate, alanine, pyruvaldehyde, malate, acetoacetate and β-hydroxybutyrate, all of which lack a 2-oxo group, were thus unable to induce HIF-1α accumulation in intact or digitonin-permeabilized cells (FIG. 2C). However, several 2-oxoacids such as ketomalonate, α-ketobutyrate, and α-ketoadipate, which contained the 1-carboxylate and 2-oxo groups, did not induce HIF-1α accumulation in intact cells suggesting that molecular features opposite from the 2-oxoacid end of the molecule were also crucial for inducing HIF-1α. Phenylpyruvate, and fluoropyruvate, which are modified on the end opposite the 2-oxoacid group, were not able to induce HIF-1α accumulation while ethyl and methyl esters of pyruvate at the 1 position were effective. Although lactate can induce HIF-1α accumulation, this requires its conversion to pyruvate via lactate dehydrogenase (Lu et al. (2002) J. Biol. Chem. 277, 23111-23115).

### Example 3

### HIF-1 Activation by 2-oxoacids is Selectively Reversed by Ascorbate

As with the basal HIF-1α expression of U87 and U251 glioma cells cultured in media, induction of HIF-1α by 3 mM pyruvate or oxaloacetate in glucose-free Krebs buffer was also completely reversed by 100 μM ascorbate (FIG. 2E). This effect was not reversed by 10 mM 2-OG, and neither ascorbate or 2-OG could reverse HIF-1α induction by hypoxia or DMOG. This selective ascorbate blockade of pyruvate and oxaloacetate inducible HIF-1α accumulation was observed in all cells used in this study (data not shown). Using U251 cells stably expressing a hypoxia regulatory element (HE)-luciferase construct (U251-HRE cells; FIG. 2E), it was found that pyruvate and oxaloacetate induced the HIF-1 regulated reporter gene as effectively as hypoxia or DMOG. Similar effects were seen using glioma cells transfected with HRE-green fluorescent protein. Induction of the HIF-1 reporter gene by the 2-oxoacids was also selectively reversed by ascorbate, but not by 2-OG. Ascorbate did not reverse HRE-luciferase activation by hypoxia or by DMOG, again suggesting a unique mode of HIF-1α regulation by pyruvate and oxaloacetate.

### Example 4

### Cysteine, Histidine, and Glutathione Antagonize Glycolytic HIF Induction

At an equivalent time in culture, it was noted that HIF-1α expression in human glioma cells tended to be higher in Krebs buffer than in complete culture media, even when the glucose concentration was identical. Thus, when U87 glioma cells cultured in MEM were changed either to Krebs buffer or to fresh MEM with identical (5.5 mM) glucose concentrations for four hours, a much higher induction of HIF-1α was always seen in Krebs buffer (FIG. 3A). Since neither Krebs buffer nor the complete MEM media contained ascorbate, this suggested to us that complete MEM contained some inhibitory activity that antagonized glycolytic activation of HIF-1α.

MEM differs from Krebs buffer by its vitamin and amino acid additives. Upon reconstitution of Krebs buffer with these additives, it was discovered that the amino acid mixture, and not the vitamin mixture, specifically blocked basal HIF-1α accumulation (FIG. 3A). The amino acid mixture also blocked the buildup of HIF-1α induced by pyruvate in cells cultured for four hours in glucose-free Krebs. By reconstituting Krebs buffer with one essential amino acid at a time at the concentration normally present in MEM it was determined that cysteine and histidine were the key amino acids responsible for inhibiting basal HIF-1α accumulation (FIG. 3B). This determination was reached following evaluation of all naturally occurring amino acids, only some of which are presented. Increasing the levels of each amino acid to 5× its normal level in MEM also revealed that cysteine and histidine were the likely endogenous inhibitors of basal HIF-1α accumulation in complete culture media. Both cysteine and its dimer cystine (Cys₂) were effective, while D-cysteine and D-histidine were not (FIG. 3C). Both amino acids lowered HIF-1α induction by glucose, pyruvate, and oxaloacetate in all cell lines used in this study (data not shown) but did not affect HIF-1α accumulation by hypoxia or the iron chelator DFO (FIG. 3C). Cysteine is involved in glutathione (GSH) synthesis. As with ascorbate, cysteine, and histidine, GSH also selectively inhibited HIF-1 (accumulation induced by pyruvate and oxaloacetate but not by hypoxia or DFO (FIG. 3C). Increasing the levels of cysteine and histidine, but not other amino acids, to 5× the level normally found in MEM, also lowered the basal expression of the HIF-1 regulated genes VEGF and GLUT3 as determined by RT-PCR (FIG. 3D). Cysteine and histidine also lowered HRE-luciferase activity induced by pyruvate or oxaloacetate in the U251-HRE cells (FIG. 3D). In addition, cells treated with oxaloacetate induce HIF-1α was shown by this western blot of nuclear extracts. Co-incubation of cells with submillimolar doses of the peptide dimer of cysteine and histidine lowers HIF-1αaccumulation (FIG. 3E). These data provide evidence for both positive and negative oxygen independent regulation of basal HIF-1 expression by cell metabolites.

### Example 5

### Pyruvate and Oxaloacetate Impede Oxygen Dependent Protein Decay

To confirm that the metabolite effects on HIF-1 were indeed oxygen independent additional experiments were performed to rule out the possibility that 2-oxoacids such as pyruvate might induce local hypoxia in cell culture through enhancement of cell respiration. Changes in mitochondrial electron transport chain activity have been shown to alter local oxygen tesions in cell culture. To address this, the hypoxia-localizing nitroimidazole compound EF-5, which forms cellular adducts only upon reductive metabolism to reactive intermediates in anaerobic environments, was used. While EF-5 adducts were clearly detected by immunofluorescence in hypoxic cells, no adducts were found under normoxia, with or without added pyruvate, despite the accumulation of HIF-1α by pyruvate (FIG. 4A) or oxaloacetate (not shown). Some reactive oxygen species can also induce HIF-1α. Since ascorbate, cysteine, histidine and glutathione all have antioxidant and/or iron reducing properties, additional experiments were performed to rule out enhanced production of oxidants such as H₂O₂ or NO by pyruvate or oxaloacetate. Despite confirming the ability of H₂O₂ and NO to induce HIF-1α accumulation in U251 cells, there was no) detection of an increase in H₂O₂ or NO levels upon treatment of U251 cells with pyruvate or oxaloacetate (FIG. 4B). Pharmacological inhibition of other signaling pathways implicated in HIF-1α stabilization, such as protein acetylation, PI3K, mTOR, and hsp70, also failed to alter HIF-1α induction by pyruvate and oxaloacetate (FIG. 4C). Next, attention was focused on HIF-1α proline hydroxylation as a target for 2-oxoacids. To explore this, experiments were performed on whether HIF-1α induced by the 2-oxoacids contained hydroxyproline in the ODD domain. This would be expected if the 2-oxoacids interfered with steps subsequent to proline hydroxylation, such as pVHL binding or proteasome activity. Using an antibody that detects hydroxylated Pro564 in the HIF-1α C-terminal ODD region hydroxyproline was detected only in HIF-1α induced by the proteasome inhibitor MG132. HIF-1α induced by pyruvate, oxaloacetate or the HPH inhibitor DMOG did not contain hydroxyproline (FIG. 4D).

Next a live cell assay for oxygen-dependent HIF-1α degradation using C6 rat glioma cells that had been stably transfected with a vector encoding the HIF-1 cc ODD region fused to GFP (ODD-GFP) was performed. The ODD-GFP protein is hydroxylated and degraded in an oxygen dependent manner similar to HIF-1α. As with HIF-1α, a basal, as well as, hypoxia-inducible accumulation of ODD-GFP has been reported. It was found that basal ODD-GFP accumulation in C6 cells, cultured in DMEM media for 24 hours, was selectively inhibited by ascorbate, while the hypoxia inducible ODD-GFP expression was not (FIG. 4E). In Krebs buffer, basal ODD-GFP accumulation was also stimulated by glucose, pyruvate, and oxaloacetate, but not by succinate. The ineffectiveness of succinate vs. pyruvate or oxaloacetate in these cells was again not due to a relative difference in cell permeability, since the same pattern was seen in digitonin-permeabilized C6 cells. Moreover, histidine and cysteine also reduced ODD-GFP induction by pyruvate or oxaloacetate. These results suggested that endogenous 2-oxoacids resembling 2-oxoglutarate could selectively block O₂ dependent protein degradation.

### Example 6

### Pyruvate and Oxaloacetate Directly Interact with HPHs

Pyruvate does not inhibit proteasomal activity, and neither pyruvate nor oxaloacetate was found to inhibit ³⁵S-pVHL binding to hydroxylated ODD (FIG. 5A). Artificial 2-OG analogs such as NOG promote HIF-1α accumulation by strongly competing for the 2-OG binding site in HPHs. To determine if pyruvate and oxaloacetate were recognized by the 2-OG binding site of HPH enzymes, a binding assay using immobilized 2-OG and in vitro translated ³⁵S-HPH homologues was developed. The observed binding of each homologue to the 2-OG column under these assay conditions was doubled by the addition of 200 μM ferrous iron (FIG. 5B). Moreover, the iron dependent binding of ³⁵S-HPHs to immobilized 2-OG was also readily displaced by addition of free 2-OG but not by succinate (FIG. 5C). Pyruvate and oxaloacetate also reversed the iron dependent binding of ³⁵S-HPHs to immobilized 2-OG (FIG. 5D), suggesting that these 2-oxoacids can interact with the HPH enzyme active site.

### Example 7

### 2-oxoacid Inhibition of HPH Activity is Ascorbate-Sensitive

To determine whether HPH activity was directly modulated by pyruvate or oxaloacetate, Pro564 hydroxylation in a biotinylated HIF-1α ODD peptide by in vitro translated HPH1, HPH2, and HPH3 was examined. The ability of streptavidin beads to pull down ³⁵S-pVHL bound to the biotinylated peptide was used as evidence of HIF-1α ODD peptide hydroxylation. This is the most specific and sensitive assay for HPH activity. Other assays, such as the ¹⁴CO₂ release assay from 1-¹⁴C 2-oxoglutarate, which are suitable for highly over-expressed HPH enzymes, can give rise to signal even in uncoupled 2-oxoglutarate decarboxylation reactions where effective substrate hydroxylation does not take place. Such assays can also give high background values in cell extracts. As exemplified by HPH-1, the ³⁵S-pVHL pulldown enzyme assay activity of each HPH homologue was dependent on 2-OG, Fe(II), and ascorbate (FIG. 5E). In the absence of ascorbate or other reducing agents, cysteine and histidine were also found to replace ascorbate in the assay and could dose dependently stimulate HIF-1α ODD hydroxylation (FIG. 5F). Some 2-OG dependent dioxygenases are able to accept pyruvate and oxaloacetate as substitutes for 2-OG while others are known to be inhibited by these 2-oxoacids. When 2-OG was replaced in the reaction mix with identical concentrations of pyruvate or oxaloacetate, no enzyme activity was detected for any of the three HPH homologues (FIG. 5G). The sustained activity of the isolated recombinant HPH enzymes required ascorbate and, as shown above, ascorbate also selectively reversed the effect of 2-oxoacids on HIF-1α stabilization. Therefore the influence of pyruvate and oxaloacetate on HPH activity over a range of ascorbate concentrations was examined Inhibition of the 2-OG supported enzymatic activity by pyruvate and oxaloacetate for all three homologues over a window of ascorbate concentrations was detected. Examples of quantitative evaluation using scintillation counting of the bound [³⁵S]-pVHL is shown in FIG. 5H. Inhibition of HPH2 and HPH3 by the 2-oxoacids was more potently reversed by ascorbate than HPH1. Ascorbate at 1 mM reversed the 2-oxoacid inhibition of all HPHs.

### Example 8

### 2-oxoacids Reversibly Inactivate Cellular HPHs

Ascorbate is required for preventing the syn-catalytic inactivation of 2-OG dependent dioxygenases. The ascorbate reversibility of pyruvate- and oxaloacetate-induced HIF-1α accumulation thus suggested that these 2-oxoacids could somehow inactivate cellular HPHs. To test this U251 cells was exposed to glucose-free Krebs buffer to either hypoxia (1% O₂) or 2 mM pyruvate for four hours. Nuclear HIF-1α protein accumulation was induced by both treatments (FIG. 6A). Then the cells were either returned to 21% O₂ or the pyruvate was removed by extensively washing with glucose-free Krebs buffer. HIF-1α immunoreactivity disappeared rapidly upon reoxygenation (return to 21% O₂) of hypoxic cells as would be expected with the resumption of oxygen-dependent HIF-1α hydroxylation and proteolysis. An entirely different pattern was seen in pyruvate treated cells. Even after extensive washing out of pyruvate, nuclear HIF-1α protein levels did not decay promptly. Persistent nuclear HIF-1α was also detected in pyruvate or oxaloacetate treated U87 cells, even 40 minutes after washout of either 2-oxoacid. However, if ascorbate, GSH, or the essential amino acids found in MEM were added to the wash, HIF-1α decay was rapidly resumed (FIG. 6B). To directly determine if the HPH activity in glioma cells was reversibly inactivated by pyruvate and oxaloacetate the ³⁵S-pVHL pulldown assay to measure HPH activity in extracts from cells treated for four hours with the 2-oxoacids was used. To capture the endogenous state of HPH activity, the cell extracts were first assayed without supplementation with exogenous reductants, ascorbate, or iron. Incubation of cells with 2 mM pyruvate or oxaloacetate clearly reduced HPH activity in the cell extracts. This reduction was not seen in cells co-treated with ascorbate (FIG. 6C). Moreover, no loss of HPH activity was seen in extracts from cells treated for four hours with hypoxia or DMOG, with or without ascorbate. The minimal HPH activity of extracts from pyruvate or oxaloacetate treated cells could also be restored by supplementation of the extracts with ascorbate or with exogenous ferrous iron (FeSO₄) during the assay (FIG. 6D).

Oxidation of HPH associated iron from the Fe(II) to the Fe(III) state has recently been proposed as a mechanism for H₂O₂ regulation of HIF-1 in cells mutated for JunD (Gerald et al. (2004) Cell 118, 781-794). It was hypothesized that NO, another potent iron oxidizer, may also work this way and observed a complete inhibition of NO induced HIF-1αaccumulation by ascorbate, Fe(II), and GSH (FIG. 6E). Addition of Fe(II) to cultured cells also prevented HIF-1α induction by pyruvate and oxaloacetate. Moreover, NO treated cells also showed sluggish HIF-1α decay, even after extensive washing (FIG. 6E) with MEM. Addition of Fe(II) but not Fe(III) to the wash accelerated the decay of NO-induced HIF-1α. A similar enhancement of HIF-1α decay by wash containing Fe(II) but not Fe(III) was seen following induction with pyruvate or oxaloacetate (FIG. 6F). The need for ferrous iron for the decay of HIF-1α during the post-induction wash was also demonstrated using DFO. Chelation of cellular iron by this agent inhibits cellular HPH activity and prevents HIF-1α decay. After washing out the DFO, addition of Fe(II) but not Fe(III) promoted decay of the accumulated HIF-1α protein. To rule out the possibility that pyruvate and oxaloacetate chelated intracellular iron a live cell fluorescence based assay for labile iron was used. Interaction of calcein trapped inside cells with iron specifically lowers its fluorescence. Iron chelators in turn increase fluorescence. Calcein loaded U251 cells displayed an increase in fluorescence when cells were challenged with the iron chelators DFO or bipyridyl and a decrease in fluorescence when Fe(II) was added (FIG. 6G). Addition of pyruvate or oxaloacetate (not shown) did not produce an increase in intracellular calcein fluorescence.

### Example 9

### Aerobic Glycolysis Regulates Basal HIF-1 Activity and Invasive Phenotype in Human Cancer Cells

Even in the presence of oxygen, most cancer cells accumulate high levels of pyruvate and lactate. In head and neck carcinomas and other human cancers high tumor lactate levels are in fact predictive of the likelihood of metastases and poor clinical outcome. Given the strong role of HIF-1 in cancer cell invasiveness it was hypothesized that the accumulation of 2-oxoacids via aerobic glycolysis could contribute to cancer cell invasiveness by inducing basal HIF-1α. To explore this two head and neck squamous carcinoma cell lines known to have disparate basal HIF-1α levels were examined. The JHU-SCC-022 cell line (022) was grown in RPMI containing 11.1 mM glucose and the UM-SCC-22B cell line (22B) was grown in DMEM containing 25 mM glucose. Media of both cell lines were supplemented equally with 10% FBS and the non-glycolytic energy source glutamine (2 mM). Both cell lines demonstrated HIF-1α accumulation under hypoxia (FIG. 7A). However, the 22B cells displayed much higher basal HIF-1 cc, elaborated higher basal levels of VEGF (FIG. 7B), and had a higher basal rate of invasion through Matrigel than the 022 cells (FIG. 7C). Serum, which activates many signaling pathways, markedly increased the invasiveness of both cell lines to a similar extent. When both cell lines were cultured in high glucose DMEM, 22B cells still consumed glucose faster and produced pyruvate and lactate at a higher rate following media change than 022 cells. This was determined by following changes in the concentration of these metabolites in media (FIG. 7 D-F).

To determine whether the endogenous accumulation of pyruvate led to inactivation of HPH activity in the 22B cells, cell extracts were prepared at progressive time points following change of media to fresh DMEM and directly monitored HIF-1 cc prolyl hydroxylation in cell extracts using the ³⁵S-pVHL capture assay as in FIG. 6C. Quantification of the captured ³⁵S-pVHL using scintillation counting revealed a progressive decline in overall HPH activity when cell extracts were not supplemented with exogenous ascorbate (FIG. 7G). Moreover, addition of 100 μM ascorbate to the cell extracts not only raised the overall ³⁵S-pVHL capture signal (not shown) but actually showed an increase in signal with increasing time in culture (FIG. 7H). Western blot analysis of these same extracts showed an increase in HPH-2 but not HPH-3 immunoreactivity (FIG. 7I).

The progressive ascorbate-reversible HPH inactivation in 22B cells was paralleled by the accumulation of ascorbate-reversible basal HIF-1α and as in other cells, ascorbate did not reverse hypoxia or DMOG stimulated HIF-1α in these cells (FIG. 7J). Furthermore, the addition of excess pyruvate (10 mM) to DMEM markedly enhanced the progressive accumulation of HIF-1α (FIG. 7K). After the 24 hours HIF-1α accumulation period the media was supplemented with either ferric or ferrous iron or ascorbate (100 μM each) and incubated the cultures for another 30 minutes. It was found that the accumulated HIF-1α was diminished to a greater extent by ferrous iron than by ferric iron while ascorbate abolished immunoreactivity. Ascorbate also lowered the basal expression of several HIF-1 regulated genes in 22B cells including carbonic anhydrase IX (CAIX), GLUT3, and matrix metalloprotease-2 (MMP-2) (FIG. 7L). Furthermore, ascorbate lowered the basal invasiveness of 22B cells through Matrigel, without affecting serum-induced invasiveness (FIG. 7M). These results implicate ascorbate-reversible, glycolysis-dependent basal HIF-1 activity in cancer progression.

The proposed sequential molecular mechanism for activity of 2-OG dioxygenases (FIG. 8A-C) first involves bidentate ligation of the apoenzyme Fe(II) complex by the 1-carboxylate and 2-oxoacid functional groups of 2-OG. Binding of the 5-carboxylate to a distinct subsite, as well as binding of the primary substrate (e.g. HIF-1α), may be also required for allosterically opening access of molecular oxygen to the axial coordination site of the ferrous iron. By properly assembling these components, the dioxygenases allow molecular oxygen to catalyze the insertion of one oxygen atom into the 2-carbon of 2-OG to form succinate and CO₂, with the other oxygen atom likely forming a ferryl intermediate that subsequently produces substrate hydroxylation. Reduced availability of the co-substrates oxygen (by hypoxia), iron (by DFO), or 2-OG (by artificial 2-OG analogs such as NOG or DMOG) is known to inhibit this family of enzymes. 2-OG dependent dioxygenases can also be syn-catalytically inactivated (FIG. 8D). This means that as a result of catalyzing iron mediated oxidations, these enzymes either become oxidized at critical amino acid residues over time or the redox state of the iron becomes useless in carrying out sustained reaction cycles. In most cases, this syncatalytic inactivation can be blocked or reversed by ascorbate (FIG. 8E). The routine observation of ascorbate and Fe(II) reversible basal HIF-1α in cancer cells suggests that either the inactivated state of HPHs is a prominent feature of malignancy or a result of the cell culture and tumor microenvironment.

In summary, it has been have shown that, in many cancer cells in culture, media glucose makes a much greater contribution to basal HIF-1α than serum derived factors. 2-oxoacid glucose metabolites has been identified as the likely mediators of this effect. Despite recent reports suggesting a link between succinate or fumarate buildup and HIF-1 activity, this method of analysis did not find a significant effect of succinate or fumarate on HIF-1α accumulation, 2-OG binding to HPHs, regulation ODD-GFP, or HRE-Luc expression. Taken together these data support a prominent role for glucose and amino acid metabolism in the regulation of HIF-1 independently from hypoxia. These data also suggest that the clinical administration of L-histidine and L-cysteine may represent an effective and innocuous adjuvant therapy for treating patients with cancer.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

TABLE 1
Incomplete list of target genes known to be upregulated
by hypoxia via the transcription factor HIF-1.

Adenylate kinase 3
α_{1β}-adrenergic receptor
Adrenomedullin
Aldolase A
Aldolase C
Carbonic anhydrase 9 (CA9)
Caeruloplasmin
Endothelin-1
Enolase 1
Erythropoietin
Glucose transporter 1 (GLUT1)
Glucose transporter 3 (GLUT3)
Glyceraldehyde-3-phosphate dehydrogenase (GAPDH)
Haeme oxygenase 1
Hexokinase 1
Hexokinase 2
Insulin-like growth factor 2 (IGF2)
IGF binding protein 1
IGF binding protein 2
IGF binding protein 3
Lactate dehydrogenase A (LDH-A)
Nitric oxide synthase 2
NTP3
p21
p35^{srj}
Phosphofructokinase L (PFK-L)
Phosphoglycerate kinase 1 (PGK-1)
Plasminogen activator inhibitor 1
Prolyl-4-hydroxylase α(I)
Pyruvate kinase M (PK-M)
Transferrin
Transferrin receptor 1
Transforming growth factor β3
Triosephosphate isomerase (TPI)
Vascular endothelial growth factor (VEGF)
VEGF receptor FLT-1

We claim:

1. A method for treating a disease associated with HIF-1α gene expression, comprising administering to said cell a composition comprising one or more agents capable of inhibiting HIF-1α activity and/or expression.

2. The method of claim 1, wherein said HIF-1α mediated gene expression includes inhibition of expression of at least one additional gene selected from the group consisting of genes encoding vascular endothelial growth factor (VEGF), glucose transporter isoform 3 (Glut-3), aldolase A (aldo A) and erythropoietin.

3. The method of claim 1, wherein the agent promotes hydroxylation of HIF-1α in said cell.

4. The method of claim 3, wherein said agents are selected from the group consisting of ascorbate, cystine, cysteine, histidine, glutathione, cytsteine-hystidine, hystidine-hystidine and derivatives thereof.

5. The method of claim 3, wherein said hydroxylation is mediated by a prolyl hydroxylase or an asparagine hydroxylase.

6. A method for inhibiting proliferation of a cancer cell in a mammal diagnosed with cancer, comprising administering to said mammal a composition comprising one or more agents which promote hydroxylation of HIF-1α.

7. The method of claim 6, wherein said agents are selected from the group consisting of ascorbate, cystine, cysteine, histidine, glutathione, cytsteine-hystidine, hystidine-hystidine and derivatives thereof.

8. The method of claim 6, wherein said method further comprises at least one additional cancer therapy.

9. The method of claim 8 wherein said additional therapy is selected from the group consisting of chemotherapies, radiation therapies, hormonal therapies and immunotherapies.

10. A method of inhibiting tissue neovascularization in a mammal comprising administering to a patient a composition comprising one or more agents which promote hydroxylation of HIF-1α.

11. The method of claim 10, wherein said agents are selected from the group consisting of ascorbate, cystine, cysteine, histidine, glutathione, cytsteine-hystidine, hystidine-hystidine and derivatives thereof.

12. The method of claim 10, wherein said tissue neovascularization is associated with cancer.

13. The method of claim 12, wherein the cancer is selected from the group consisting of breast, ovary, melanoma, prostate, colon and lung.

14. The method of claim 12, wherein said method further comprises at least one additional anti-angiogenic agent.

15. The method of claim 10, wherein said tissue neovascularization is associated with inflammatory conditions.

16. The method of claim 15, wherein said inflammatory condition is selected from the group consisting of dermatitis, psoriasis, arthritis.

17. The method of claim 16 wherein said arthritis is rheumatoid arthritis.

18. The method of claim 15, wherein said method further comprises at least one additional anti-inflammatory agent.

19. The method of claim 10, wherein said tissue neovascularization is associated with loss of vision.

20. The method of claim 19, wherein said loss of vision caused by retinal neovacularization.

21. The method of claim 20 wherein said retinal neovacularization caused by diabetic retinopathy, macular degeneration or sickle cell retinopathy.

22. The method of claim 21 wherein said macular degeneration is the wet form.

23. The method of claim 21 wherein said macular degeneration is the dry form.

24. A method of inhibiting tissue neovascularization in a mammal associated with increased levels of VEGF in a mammal by administering to said patient a composition comprising one or more agents which promote hydroxylation of HIF-1α.

25. The method of claim 24, wherein said agents are selected from the group consisting of ascorbate, cystine, cysteine, histidine, glutathione, cytsteine-hystidine, hystidine-hystidine and derivatives thereof.

26. The method of claim 24, wherein said method further comprises at least one additional anti-VEGF agent.
